(19) United States
(12) Patent Application Publication
(10) Pub. No.: US 2011/0077198 A1
(43) Pub. Date: Mar. 31, 2011

(54) COMPOSITIONS AND METHODS FOR INHIBITING THE ACTIVATION OF DSRNA-DEPENDENT PROTEIN KINASE AND TUMOR GROWTH INHIBITION

(76) Inventors: Michael John Tisdale, Warwickshire (GB); Helen Laura Eley, Kent (GB); Steve Thomas Russell, West Midlands (GB); Kevin Burke Miller, Minneapolis, MN (US)

(21) Appl. No.: 12/743,312

(22) PCT Filed: Oct. 3, 2008
(86) PCT No.: PCT/US08/78667

§ 371 Date: Dec. 2, 2010

Related U.S. Application Data
(60) Provisional application No. 60/990,134, filed on Nov. 26, 2007.
(60) Provisional application No. 61/037,371, filed on Mar. 18, 2008.

(51) Int. Cl. A61K 38/18; A61K 38/00; A61K 31/70; A61K 31/7088; A61K 31/353; A61K 31/20; A61K 31/198; A61P 17/00; A61P 35/00; A61P 11/00; A61P 9/00; A61P 3/10; A61P 37/00; A61P 31/18

(52) U.S. Cl. 514/8.9; 514/1.1; 514/23; 514/44 R; 514/456; 514/558; 514/561; 514/564; 514/565

(57) ABSTRACT

The present invention relates to compositions and methods for preventing and treating a condition in a mammalian subject that include at least one inhibitor of double stranded RNA dependent protein kinase (PKR-I) prior to or concurrently with the treatment, wherein the treatment results to an inhibition of activation of dsRNA-dependent protein kinase. The compositions and methods of the present invention further include at least one potentiator that further enhances the inhibition of phosphorylation by PKR-I.

### TECHNICAL FIELD

The present invention relates to compositions and methods for preventing and treating a condition in a mammalian subject that includes at least one inhibitor of double stranded RNA dependent protein kinase (PKR-I) prior to or concurrently with the treatment, wherein the treatment results in an inhibition of activation of dsRNA-dependent protein kinase. The compositions and methods of the present invention further include at least one potentiator that further enhances the inhibition of phosphorylation by PKR-I.

### BACKGROUND OF THE INVENTION

Cachexia is commonly associated with a number of disease states, including acute inflammatory processes associated with critical illness and chronic inflammatory diseases, such as cancer, sepsis, congestive heart failure, rheumatoid arthritis, chronic obstructive pulmonary disease, and human immunodeficiency virus infection. It is also associated with other known muscle wasting diseases and disorders, e.g., sarcopenia, an age-related loss of muscle mass. Cachexia is responsible for the deaths of 10%-22% of all patients with cancer and approximately 15% of the trauma deaths that occur from sepsis-induced organ dysfunction and malnutrition days to weeks after the initial traumatic event.

Cancer patients, particularly those of the gastrointestinal tract, exhibit progressive skeletal muscle wasting or cachexia, which, in turn, reduces their quality of life and survival time. Cancer cachexia in a patient is characterized by anorexia, weight loss, premature satiety, asthenia (a feeling of weakness without actual loss of strength), loss of lean body mass, and multiple organ dysfunction. A loss of lean body mass associated with cancer cachexia not only weakens the individual and makes activities of daily living difficult, but can weaken the patient to the point that they do not have the strength to undergo chemo-and/or radiation therapy.

Cachexia is due to a combination of depressed protein synthesis (hypoanabolism) and elevated endogenous protein breakdown (catabolism), with the oxidation of resultant amino acids (O'Keefe, S. J. D. et al., Cancer Res., 50:1226-1230, 1990). The mechanism for increased protein degradation has been attributed to an increased expression of the ubiquitin-proteasome proteolytic pathway. Khal, J. et al., Int. J. Biochem. Cell. Biol., 37:2196-2206, 2005. The mechanism underlying the failure to maintain protein synthesis in cancer cachexia remains unknown. However, it was until recently that a mechanism has been proposed that may explain the depression in protein synthesis and increased degradation of myofibrillar proteins in cachexia. This mechanism involves the activation of double-stranded RNA-dependent protein kinase (PKR) via autophosphorylation. Activation of PKR by agents such as PIF (proteolysis-inducing factor) and Ang II (angiotensin II) induces phosphorylation of eukaryotic initiation factor 2α (eIF2α), leading to inhibition of translation initiation, through competition with the guanine-nucleotide exchange factor, eIF2B, which prevents the conversion of the conversion of eIF2 from its GDP-bound state into the active GTP bound form. Russell, S. T. et al., Cell. Signalling, 19:1797-1806, 2007.

### Regulation of Protein Synthesis Via Translation Initiation

The regulation of translation initiation involves (i) the binding of initiator methionyl-transfer RNA (met-tRNA) to the 40s ribosomal subunit; and (ii) the binding of mRNA to the 43s pre-initiation complex. During the first step, met-tRNA binds to the 40s ribosomal subunit as a ternary complex with eukaryotic initiation factor 2 (eIF2) and guanosine triphosphate (GTP). This step is followed by the hydrolysis of GTP to guanosine diphosphate (GDP) with eIF2 release from the ternary complex. The eIF2 must exchange the GDP for GTP to involve in another round of initiation. This takes place through the action of another eukaryotic initiation factor 2, eIF2B, which mediates guanine nucleotide exchange on eIF2. eIF2B is regulated by eIF2B phosphorylation of eIF2 on its alpha subunit, which converts it from a substrate unto a competitive inhibitor of eIF2B.

In the second step, the binding of mRNA to the 43s pre-initiation complex requires a group of protein collectively referred to as eIF4F, a multi-subunit complex consisting of eIF4A (an RNA helicase), eIF4B (which functions in conjunction with eIF4A to unwind secondary structure in the 5′ untranslated region of the mRNA), eIF4E (which binds to the m7GTP cap present at the 5′ end of the mRNA), and eIF4G (which functions as a scaffold for eIF4E, eIF4A and the mRNA). Together, the eIF4F complex serves to recognize, unfold, and guide the mRNA to the 43s pre-initiation complex. The availability of the eIF4E for the eIF4F complex formation appears to be regulated by the translational repressor eIF4E-binding protein 1 (4E-BP1). The 4E-BP1, in turn, competes with the eIF4G to bind eIF4E and is able to sequester eIF4E into an inactive complex. The binding of 4E-BP1 is regulated through phosphorylation by the kinase mammalian target of rapamycin (mTOR), where increased phosphorylation causes a decrease in the affinity of 4E-BP1 for eIF4E.

Induction of the ubiquitin-proteasome pathway by PIF and Ang II requires the activation of the transcription factor nuclear factor-κB (NF-κB). Wyke, S. M. and Tisdale, M. J., Br. J. Cancer, 92:711-721, 2005. PKR has been shown to activate the upstream kinase IκB kinase that would result to the degradation of the inhibitor protein IκB. The degradation of IκB would, in turn, lead to NF-κB release. The released NF-κB would migrate to the nucleus that would result to transcriptional activation of specific genes (Zamanian-Daryoush, M. et al., Mol. Cell. Biol., 20:1278-1290, 2000). Myotubes containing mutant PKR failed to activate NF-κB in response to either PIF or Ang II and also failed to induce the ubiquitin-proteasome pathway. These results suggested that NF-κB activity is needed for the induction of the ubiquitin-proteasome pathway by PKR.

### Amino Acids

Nine of the twenty amino acids are considered essential in humans, as the body cannot make them. These nine amino acids must be obtained through the diet of the individual. A deficiency of one or more of the amino acids can cause a negative nitrogen balance, wherein more nitrogen is excreted than is ingested as proteins are degraded faster than they are being made, which may lead to the disruption of enzymatic activity and loss of muscle mass.

Anabolic factors, such as insulin, insulin-like growth factors and amino acids are known to increase protein synthesis and cause muscle hypertrophy. Branched-chain amino acids, particularly leucine, can initiate signal transduction pathways, which often include mTOR and eIF2, that modulate translation initiation. As an example, an amino acid starvation would lead to an increase in eIF2-α phosphorylation and a depression in protein synthesis.

In patients with cachexia, there is a general decrease in the plasma levels of free amino acids. The maximum decrease is often found for the branched-chain amino acids (BCAAs), such as leucine, isoleucine, and valine. BCAAs, comprise 14-18% of the total amino acids in muscle proteins, function as building blocks and modulators of protein synthesis. Of the three BCAA mentioned herein, leucine is most potent in stimulator of muscle protein synthesis, while the remaining two are less effective. The mechanism for stimulating protein synthesis, as reported by Anthony, J. C. et al. (J. Nutr., 130:139-145, 2000), is via the activation of the mRNA binding steps in translation initiation through hyperphosphorylation of 4E-BP1 (eIF4E-binding protein 1), which, in turn, results to in the release of eIF4E from the inactive 4E-BPI-eIF4E complex. The released eIF4E then associates with eIF4G to form the active eIF4F complex. The increase formation of the eIF4F complex promotes the migration and recruitment of 43 S pre-initiation complex to the mRNA, enhancing peptide chain initiation.

Although the effect of BCAAs on PKR activation has not been fully studied, Eley and her colleagues recently reported that BCAAs, such as leucine and valine, significantly suppress the loss of body weight of mice bearing a cachexic-inducing tumor (MAC-16), which resulted to significant increase in skeletal muscle wet weight through an increase in protein synthesis and a decrease in degradation. Eley, H. L. et al., Biochem J., 407(1):113-120, 2007. This effect of leucine on PKR phosphorylation appears to be due to an increased expression of PPI (protein phosphatase I), which has been shown to bind to the N-terminal regulatory region of PKR and inhibit autophosphorylation (Tan, S. L. et al., J. Biol. Chem., 277:36109-36117, 2002. This study by Eley and her colleagues is the first report to show that leucine can attenuate PKR and eIF2α phosphorylation in skeletal muscles of MAC-16 tumor-bearing mice and in murine myotubes when exposed to PIF. The concentration of leucine employed in vitro (2 mmole/l) is the same as that reported previously by Anthony et al. (J. Nutr., 130:2413-2419, 2000) in serum of rats when leucine was administered at 1.35 g/kg of body weight.

On the other hand, weight loss in mice-bearing the MAC-16 tumor, as reported by Eley and her colleagues, was associated with an increased amount of eIF4E bound to its binding protein, 4E-BPI and a progressive decrease in the active eIF4G-eIF4E complex due to hypophosphorylation of 4E-BPI. This may be attributed to a reduction in the phosphorylation of mTOR (mammalian target of rapamycin), which may be responsible for the decreased phosphorylation of p70^{S6k} (70 kDa ribosomal S6 kinase). A 5-fold increase in the eEF2 (eukaryotic elongation factor 2) was noted, which also decreases protein synthesis via a decrease in translation elongation. Treatment of leucine reverses this effect by (1) increasing mTOR and p70^{S6k}; (2) causing hyperphosphorylation of 4E-BPI; (3) reducing the amount of 4E-BPI associated with eIF4E; (4) causing an increase in the formation of eIF4G-eIF4E complex; and (5) reducing eIF2α phosphorylation and PKR activation to cause an increase in protein synthesis and attenuation of increased protein degradation, respectively.

Based on the above, a combination of an inhibitor to PKR and nutritional supplements such as branched-chain amino acids can be employed together to treat and prevent cancer cachexia or other disease-associated with cachexia.

The inventors, in the meantime, have recently described their work in a PCT publication, WO/2007/064618, relating to the administration of one or more branched-chain amino acid (BCAA), a BCAA precursor, a BCAA metabolite, BCAA-rich protein, protein manipulated to enrich the BCAA content or any combination thereof in the treatment of muscle loss in a mammal. Nutritional formulations suitable for such administration were also described.

The prevention and treatment of cachexia and anorexia remain an existing problem to the medical community. Nutritional supplemental support to replete loss of muscle mass in the cancer or any disease-bearing host remains largely ineffective. Thus, there remains a need of improvements in clinical approaches to enhance the efficacy of chemotherapy or any form of cytotoxic anti-neoplastic therapy with the combined application of nutrition and chemotherapy.

### SUMMARY OF THE INVENTION

The present invention relates to compositions and methods for treating a condition that includes at least one inhibitor of double stranded RNA dependent protein kinase (PKR-I) in a mammal prior to or concurrently with a treatment, wherein the treatment results in an inhibition of activation of dsRNA-dependent protein kinase. In addition, the compositions and methods of the present invention further include at least one potentiatior, wherein at least one potentiator enhances the inhibition of phosphorylation by the PKR-I in the mammal. Furthermore, the present invention relates to composition and methods of enhancing the efficacy of chemotherapeutic agents in treating or improving cancer conditions, autoimmunity or other disorder for which chemotherapeutic agents are used, wherein at least one PKR-I is used with or without at least one nutritional compound.

In one feature of the invention, the condition may include, but is not limited to, cancer, an inflammatory disease, sepsis, congestive heart failure, rheumatoid disorders, including, but not limited to ankylosing sponylitis, fibromyalgia, rheumatic organ disease (i.e., heart, lung, kidney and vasculitis), lupus including systemic lupus erythematosus, temporal arteritis and polymyalgia rheumatica, Sjorgren's syndrome, rheumatoid arthritis, chronic obstructive pulmonary disease, a neurodegenerative disease, an autoimmune disease, a human immunodeficiency virus infection, immunity-related conditions including, but not limited to allergic conditions, asthmatic conditions and those related to transplant, graft or transfusion, diabetes, psoriatic disorders, a skin disease, cellular aging, Cushing Disease, rheumatic fever, and progeria.

In another feature of the invention, at least one of the potentiator, together with PKR-I, enhances the improvement or reduction of the severity of one of the above-mentioned conditions in the affected mammal.

In yet another feature of the invention, the PKR-I may be natural or synthetic and may be enterally or parenterally administered either alone or in combination with at least one potentiator. The route of the parenteral administration is subcutaneous, intravenous, intramuscular or topical. As for the enteral administration, it may either through intranasal, intraoral, nasogastric, orogastric or via a gastric port, a jejunal port or an ileal port.

In another feature of the invention, the composition is a nutritional composition. The potentiator may be an inhibitor to PKR, an analog of PKR-I, a phosphorylation inhibitor of PKR, a chemotherapeutic agent, an angiogenic agent, a vasodilatory agent, a catechin-flavanol, a bioactive protein, a branched-chain amino acid, an essential amino acid, an amino acid, an amino acid analog, a nucleotide, a vitamin, a glutamine, a sialic acid oligosaccharide, an L-theanine, a prebiotic, a probiotic, a synbiotic, an essential fatty acid, a PUFA, an MUFA, and an anti-oxidant. The potentiator may be at least one L-glutamine agonist, e.g., L-theanine. The nucleotide may be an RNA, e.g., adenine, guanine, uracil, or cytosine. An example of a chemotherapeutic agent is 5-Fluorouracil or gemcitabine. An example of an amino acid may be Norleucine, arginine, L-citrulline, L-theanine or glutamine. A bioactive agent may be a TGF-β1, TGF-β2, TGF-β3, TGF-β4 or TGF-β5.

The PKR-I may function as an inhibitor of cell growth or cell replication in the mammal. The treatment may either be in form of radiotherapy or chemotherapy.

The compositions of the present invention may further include at least one modifier of Protein Phosphatase-1α (PP1-A), wherein PP1-A dephosphorylates the phosphorylated forms of PKR. In addition, at least one modifier is a branched-chain amino acid that is a leucine, isoleucine or valine.

The present invention also includes the methods for treating a condition in a mammal that include administering to the mammal the compositions as described hereinabove, wherein the treatment results to an inhibition of activation of dsRNA-dependent protein kinase in the treated mammal.

Other features and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

These and other features of the present invention will be more readily understood from the following description of the various aspects of the invention taken in conjunction with the accompanying drawings that depict various embodiments of the invention, wherein:

FIG. 1 is a diagram showing the pathways leading to a depression of protein synthesis and an increase in protein degradation in skeletal muscle via PKR activation.

FIG. 2 shows the effect of increasing concentrations of the PKR inhibitor on growth of the MAC16 (♦) and MAC13 (▪) tumors in vitro. The experiment was repeated three times. Differences from control are indicated as c, p<0.001.

FIGS. 3A-3B presents Western blotting showing expression of phosphor and total forms of PKR (3A) and eIF2α (3B) in MAC16 (lanes 1 to 3) and MAC13 tumors (lanes 4 to 6). The densitometry analysis shows the ratio of the phosphorylated (pHs) to total(tot) forms, and represents the average of three separate Western blots. Differences from the MAC16 tumor are shown as b, p<0.01 or c, p<0.001.

FIGS. 4A-4B shows the effect of treatment of mice bearing the MAC16 tumor with either solvent (DMSO:PBS, 1:20) control (lanes 1 to 3) or the PKR inhibitor at concentrations of 1 (lanes 4 to 6) or 5 (lanes 7 to 9) mgkg-1, administered daily by sac. injection (Eley et al, 2007) on phosphorylation of PKR (4A) and eIF2α (4B). The number of mice in each group n=6. The densitometry analysis shows the ratio of phosphorylated (pHs) to total (tot) forms, and represents the average of three Western blots. Differences from control are shown as c, p<0.001.

FIGS. 5A-5E presents the effect of concentration of the PKR inhibitor on autophosphorylation of PKR (5A and 5B) and expression of the 20S protease α-subunits (5C and 5D) in MAC16 (5A and 5C) and MAC13 (5B and 5D) cells. The densitometry analysis shows the ratio of phosphorylated (pHs) to total (tot) forms, and represents the average of three separate Western blots. Differences from control are shown as a, p<0.05, b, p<0.01 or c, p<0.001. (5E) Relationship between expression of 20S protease α-subunits measured densitometrically in MAC16 cells treated with the concentrations of the PKR inhibitor shown in (5C) and the levels of phosphorylated PKR shown in (5A). The correlation coefficient is 0.957.

FIG. 6 shows the protein synthesis in MAC13 and MAC16 cells in vitro over a 4 h period as described in Methods section. Difference from the MAC16 tumor is shown as c, p<0.001.

FIGS. 7A-7B shows nuclear accumulation of NF-κB in MAC16 and MAC13 tumors (7A) and (7B) in the MAC16 tumor from mice treated with the PKR inhibitor at 5 mgkg-1 for 4 days or solvent control, as determined by EMSA. The densitometric analysis represents the average of three separate blots. Differences from the MAC16 tumor in (7A) is shown as b, p<0.01, while differences from the solvent control in (7B) is shown as c, p<0.001.

FIG. 8 presents the effect of 5FU alone at 0 (▪), 1 (□), 2.5 ( [symbol] ), 5 ( [symbol] ) and 10 μM ( [symbol] ), or in combination with the PKR inhibitor (PKR) at 100 and 200 nM on growth of MAC16 cells in vitro, and effect of gemcitabine at 0 (▪), 3.8 (□), 9.5 ( [symbol] ), 19 ( [symbol] ) and 38 μM ( [symbol] ) alone or in combination with the PKR inhibitor on growth of MAC16 cells. The effect of the PKR inhibitor alone at 100 and 200 nM is also shown. Differences from control are shown as b, p<0.01 or c, p<0.001, while differences in the presence of the PKR inhibitor are shown as e, p<0.01 or f, p<0.001.

### DETAILED DESCRIPTION OF THE PREFERRED EMBODIMENTS

The present invention relates to methods of enhancing the efficacy of chemotherapeutic agents in treating cancer with the use of an inhibitor of double-stranded RNA Protein Kinase (PKR-I) with or without nutritional compounds.

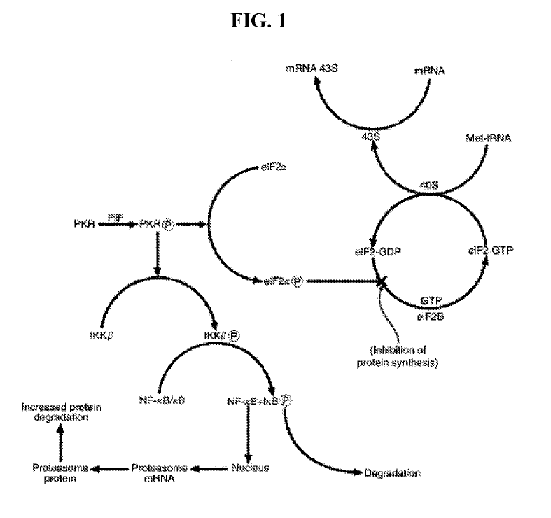

By using an inhibitor of PKR, the depression in protein synthesis was completely attenuated and the induction of eIF2α phosphorylation was prevented (see also FIG. 1). The PKR inhibitor also attenuated the depression of protein synthesis induced by both PIF and Ang II and prevented the increase in proteasome expression and activity in both murine and human models of cachexia. The proposed mechanism elucidating the depression of protein synthesis and an increase in protein degradation in muscle cachexia via PKR autophosphorylation is summarized in FIG. 1. Eley, H. L. and Tisdale, M. J., J. Biol., 282:7087-7097, 2007; Eley, H. L. and Tisdale, M. J., Br. J. Cancer, 96:1216-1222, 2007; and Eley, H. L. et al., Br. J. Cancer, 98(2):443-449, 2008. Based on these findings, the inhibitors of PKR may be used to therapeutically prevent muscle atrophy in cancer patients and also in other cachexia-associated diseases.

For example, as observed by the inventors, the levels of both phosphorylated forms of PKR and eIF2α were greatly enhanced in muscle of human cancer patients having weight loss irrespective of their amounts. A linear relationship was noted between the phosphorylation of PKR and eIF2α, which led to the suggestion that PKR phosphorylation resulted to eIF2α phosphorylation. However, the levels of myosin decreased as the level of weight loss increased. A similar linear relationship between myosin expression and the extent of eIF2α phosphorylation. These findings suggest that PKR phosphorylation may be an important initiator of muscle wasting in cancer patient. Eley, H. L. et al., Br. J. Cancer, 98(2):443-449, 2008.

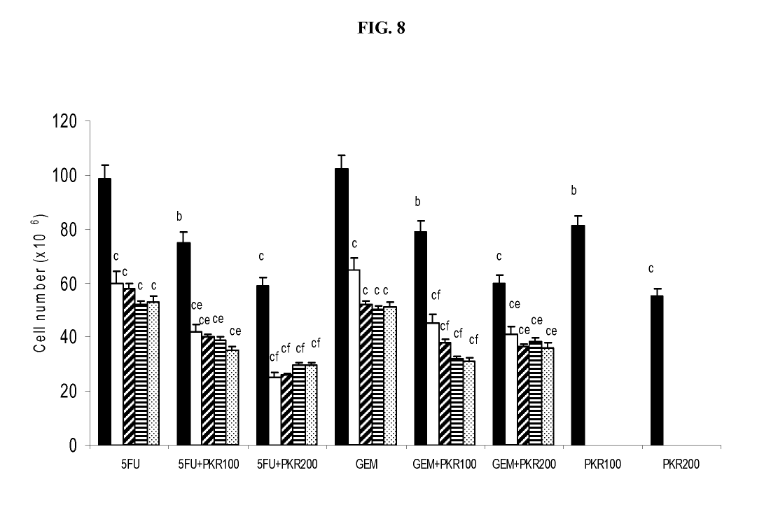

Without limiting the present invention to any particular mechanism, the inventors of the present invention have found that administration of a PKR-I reduces the growth of tumor cells more effectively in combination with chemotherapeutic agent (e.g., 5-fluorouracil or gemcitabine) than when either was used alone. As a consequence, the administration of PKR-I may be a direct or indirect in its ability to potentiate chemotherapy. Both 5-fluorouracil or gemcitabine are chemotherapeutic compounds are commonly used in treating neoplastic growth (e.g., colon cancer) Without being bound by theory, it is believed that PKR-I reduces the growth of cancer cells when introduced at very specific concentrations (maximal effect at 200 nM, diminished the effect at lower and greater concentrations). In addition, the inhibition of PKR further decreased the proliferation of cancer cells exposed to chemotherapeutic drugs. The cellular inhibition appeared to be a synergistic effect as compared to the inhibition observed with either compound alone (see FIG. 8). The administration of specific nutritional compounds, structurally unrelated to the PKR I compounds, is believed to also reduce cancer cell growth and prevent cancer cachexia. However, the nutritional compounds behave through a different mechanism than the PKR-I compounds that were previously described by Jammi et al., Biochem. Biophys. Res. Commun., 308:50-57, 2003.

As used herein, the term “potentiator” or “potentiate” relates to a compound or an agent which when used in combination with another agent and/or a nutritional compound produces a synergistic effect of both agents/compounds, being greater than the sum of the effects of each used alone. According to the present invention, a potentiator may include, but not limited to, an inhibitor to PKR, an analog of PKR-I, a phosphorylation inhibitor of PKR, a nutritional supplement or compound, a chemotherapeutic agent, an angiogenic agent, a vasodilatory agent, a catechinflavanol, a bioactive protein, a branched-chain amino acid, an essential amino acid, an amino acid or amino acid analog, a nucleotide or RNA, a vitamin, a glutamine, a sialic acid oligosaccharide, an L-theanine, a prebiotic, a probiotic or a synbiotic, an essential fatty acid, a PUFA and/or MUFA, and an anti-oxidant.

As used herein, the terms “treatment” and “treat” refers to both prophylactic or preventive treatment and curative or disease-modifying treatment, including treatment of patients at risk of contracting a disease or suspected to have contracted a disease, as well as patients who are ill or have been diagnosed as suffering from a disease or medical condition. These terms also refer to the maintenance and/or promotion of health in an individual not suffering from a disease but who may be susceptible to the development of an unhealthy condition, such as nitrogen imbalance or muscle loss. Consequently, an “effective amount” is an amount that treats a disease or medical condition in an individual or, more generally, provides a nutritional, physiological or medical benefit to the individual. In addition, while the terms “individual” and “patient” are often used herein to refer to a human, the present invention is not limited. Accordingly, the terms “individual” and “patient” refer to any animal that can benefit from the treatment.

### Cachexia

Cachexia or wasting is a condition of severe malnutrition and negative nitrogen balance characterized by anemia (drop in hemoglobin), anorexia (lack or severe loss of appetite), weight loss, and muscle atrophy. The physiological, metabolic, and behavioral changes in cachexia are associated with patient complaints of weakness, fatigue, gastrointestinal distress, sleep/wake disturbances, pain, listlessness, shortness of breath, lethargy, depression, malaise and the fear of being burdensome on family and friends. Cachexia is seen in several diseases including but are not limited to, AIDS, cancer, post hip fracture, chronic heart failure, chronic lung disease such as chronic obstructive lung disease and chronic obstructive pulmonary disease, liver cirrhosis, renal failure, autoimmune diseases such as rheumatoid arthritis and systemic lupus, sepsis, tuberculosis, cystic fibrosis, Crohn's disease and sever infection. Besides these chronic infections and malignant conditions, cachexia has also been identified in patients after extensive traumatic injury and in aging persons with failure to thrive syndrome.

Two main components contribute to cancer cachexia: (1) a loss of appetite and (2) a metabolic response to stress that causes a preferential loss of muscle at a rate greater than would be expected from the lack of nutritional intake alone. Consequently, a nutritional supplement to ameliorate the rate of loss of muscle mass in patients with cancer would have an important clinical impact.

Cancer cachexia is not simply a local effect of the tumor. Alterations in protein, fat, and carbohydrate metabolism occur commonly. For example, abnormalities in carbohydrate metabolism include increased rates of total glucose turnover, increased hepatic gluconeogenesis, glucose intolerance and elevated glucose levels. Increased lipolysis, increased free fatty acid and glycerol turnover, hyperlipidemia, and reduced lipoprotein lipase activity are often observed. 0 The weight loss associated with cancer cachexia is caused not only by a reduction in body fat stores but also by a reduction in total body protein mass, with extensive skeletal muscle wasting. Increased protein turnover and poorly regulated amino acid oxidation may also be important. The presence of host-derived factors produced in response to the cancer have been implicated as causative agents of cachexia, e.g., tumor necrosis factor-α (TNF-α) or cachectin, interleukin-1 (IL-1), IL-6, γ-interferon (γ-IFN), and prostaglandins (PGs; for example, e.g., PGE₂).

Weight loss is common in patients with carcinomas of the lung and gastrointestinal tract, resulting in a massive loss of both body fat and muscle protein, while non-muscle protein remains unaffected. While loss of body fat is important in terms of energy reserves, it is loss of skeletal muscle protein that results in immobility, and eventually impairment of respiratory muscle function, leading to death from hypostatic pneumonia. Although cachexia is frequently accompanied by anorexia, nutritional supplementation alone is unable to maintain stable body weight and any weight that is gained is due to an increase in adipose tissue and water rather than lean body mass. Double stranded (ds) RNA dependent protein kinase (PKR)

As used herein, the term “PKR” refers to a protein having the function of, and alternatively referred to as, the proteins: “double-stranded RNA dependent protein kinase,” double-stranded RNA dependent eIF-2α kinase,” “DAI” (Jimenez-Garcia, et al., J. Cell Sci. 106:11-12, 1993), “dSI,” “p68 (human) or p65 (murine) kinase” (Lee, et al., J. Interferon Cytokine Res. 16:1073-1078, 1996), or dsRNA-PK. See also, Clemens, et al., J. Interferon Res. 13:241, 1993. PKR is the only identified dsRNA-binding protein known to possess a kinase activity. PKR is a serine/threonine kinase, whose enzymatic activation requires dsRNA binding and consequent autophosphorylation (Galabru, J. & Hovanessian, A., J. Biol. Chem. 262:15538-15544, 1987; Meurs, E. et al., Cell, 62:379-390, 1990). The best characterized in vivo substrate of PKR is the α subunit of eukaryotic initiation factor-2 (eIF-2α) which, once phosphorylated, leads ultimately to inhibition of cellular and viral protein synthesis (Hershey, J. W. B., Ann. Rev. Biochem. 60:717-755, 1991). This particular function of PKR has been suggested as one of the mechanisms responsible for mediating the antiviral and anti-proliferative activities of IFN-α and IFN-β. An additional biological function for PKR is its putative role as a signal transducer. Kumar et al. demonstrated that PKR can phosphorylate IκBα, resulting in the release and activation of nuclear factor-κB (NF-kB) (Kumar, A. et al., Proc. Natl. Acad. Sci. USA 91, 6288-6292, 1994). Given the well-characterized NF-kB site in the IFN-b promoter, this may represent a mechanism through which PKR mediates dsRNA activation of IFN-b transcription (Visvanathan, K. V. & Goodbourne, S., EMBO J., 8, 1129-1138, 1989).

Activation of PKR involves two molecules binding in tandem to double stranded RNA and then phosphorylating each other in an intramolecular event. (Wu et al. 1997, J. Biol. Chem. 272:1291-1296). PKR has been implicated in processes that rely on apoptosis as control mechanisms in vivo including antiviral activities, cell growth regulation and tumorigenesis (Donze et al. EMBO J., 14: 3828-3834, 1995; Lee et al., Virology, 199:491-496, 1994; Jagus et al. Int. J. Biochem. Cell. Biol. 1989, vol. 9: 1576-86).

### PKR Inhibitors (PKR-Is)

PKR is involved in a variety of cellular processes, including signal transduction, differentiation, and apoptosis. Inhibitors of PKR (PKR-I) may be used, according to the present invention, to treat disorders associated with abnormal cellular responses, e.g., neurodegenerative disorders (e.g, Huntington diseases, Alzheimer's disease, and Parkinson's disease). PKR inhibitors that may be suitable for use in the compositions, kits, and methods of the invention include those described in Shimazawa et al., Neurosci. Lett., 409:192-195, 2006, Peel, J. Neuropathol. Exp. Neurol., 63:97-105, 2004, Bando et al., Neurochem. Int., 46:11-18, 2005, Peel et al., Hum. Mol. Genet., 10:1531-1538, 2001, and Chang et al., J. Neurochem. 83:1215-1225, 2002.

Analogs of PKR-I may also include but are not limited to 2-aminopurine (2-AP), 9-(4-bromo-3,5-dimethyl-pyridin-2-yl)-6-chloro-9H-purin-2-ylamine, 9-(4-bromo-3,5-dimethyl-pyridin-2-ylmethyl)-6-chloro-9H-purin-2-ylamine, phosphate salt, 9-(4-bromo-3,5-dimethyl-pyridin-2-ylmethyl)-6-chloro-9H-purin-2-ylamine, hydrochloric acid salt, 6-bromo-9-(4-bromo-3,5-dimethyl-pyridin-2-ylmethyl)-9H-purin-2-ylamine, 6-bromo-9-(4-bromo-3,5-dimethyl-1-oxy-pyridin-2-ylmethyl)-9H-purin-2-ylamine, 2-(2-amino-6-chloro-purin-9-ylmethyl)-3,5-dimethyl-pyridin-4-ol, 9-(4-allyloxy-3,5-dimethyl-pyridin-2-ylmethyl)-6-chloro-9H-purin-2-ylamin-e, 6-chloro-9-[4-(2-ethoxy-ethoxy)-3,5-dimethyl-pyridin-2-ylmethyl]-9H-pur-in-2-ylamine, 6-chloro-9-(4-cyclopropylmethoxy-3,5-dimethyl-pyridin-2-ylmethyl)-9H-purin-2-ylamine, 6-chloro-9-(4-isobutoxy-3,5-dimethyl-pyridin-2-ylmethyl)-9H-purin-2-ylamine, 6-chloro-9-(4-chloro-3,5-dimethyl-pyridin-2-ylmethyl)-9H-purin-2-ylamine, 6-chloro-9-(3,5-dimethyl-pyridin-2-ylmethyl)-9H-purin-2-ylamine, and 6-bromo-9-(4-methoxy-3,5-dimethyl-pyridin-2-ylmethyl)-9H-purin-2-ylamine, phosphate salt. PKR inhibitor analogs are described in Jammi et al., Biochem. Biophys. Res. Commun., 308:50-57, 2003 (Calbiochem Cat. No. 527450).

The term “PKR expression” refers to transcription and translation of a PKR encoding nucleic acid sequence, the products of which include precursor RNA, mRNA, polypeptide, post-translation processed polypeptide, and derivatives thereof, and including PKRs from other species such as murine or simian enzymes. By way of examples, assays for PKR expression include autophosphorylation assays (Der and Lau, Proc. Natl. Acad. Sci. USA, 92:8841-8845, 1995), assay for eIF2α phosphorylation (Zamanian-Daryoush, M. et al., Oncogenes, 18:315-326, 1999), a kinase assay carried out by immunoprecipitation of PKR and in vitro assay for kinase (Zamanian-Daryoush, M. et al., Mol. Cell. Biol., 20:1278-1290, 2000). Exemplary assays and for PKR expression and/or production include protein assays such as Western blot and assays for PKR mRNA such as reverse transcriptase polymerase chain reaction (RT-PCR) assays, Northern blot analysis, dot blot analysis or in situ hybridization analysis using appropriately labeled probe based on PKR-encoding nucleic acid sequence.

As mentioned earlier, PKR is a protein that phosphorylates a series of other cellular proteins involved in the breakdown of proteins in the body or cell. These proteins, targeted for degradation, are not limited to striated muscle proteins, but also include cellular proteins that are either structural or regulatory (e.g., enzymes and signaling proteins, actin filaments, etc). As a result, inhibition of the PKR protein has been shown to alter the mechanism that controls degradation of cellular proteins. PKR-inhibition is expected to interfere with normal protein metabolism and limit both the degradation and synthesis of new proteins.

The uses and benefits derived from the administration of PKR-I according to the present invention are discussed hereinbelow:

In cancer therapy applications—Because PKR-inhibitors (PKR-I) have been shown to inhibit protein degradation associated with ubiquitin-mediated proteosomal pathway, PKR-I is believed to reduce replication of tumor cells and thus slow tumor growth, as evidenced by the Examples provided herein. The use of PKR-I may also promote a reduction in tumor cell numbers. The mechanism of tumor inhibition is not fully elucidated, but may include interference with proteins that control the cell replication cycle as well as intracellular proteins necessary to maintain cellular integrity.

As previously reported, PKR-I may be used to inhibit protein degradation of skeletal muscle that is often upregulated in cancer cachexia. Cancer cachexia typically results in a very rapid loss of lean muscle tissue thus increasing the patients risk of mortality.

In contrast to systemic administration, the local injection of PKR-I into a tumor is believed to retain normal skeletal muscle metabolism (which includes protein breakdown) while limiting the growth of tumor cells through limiting intracellular protein metabolism.

In autoimmune diseases—Hyperinflammation often results in the production of excess proteins that regulate the inflammatory response. Because hyperinflammation is directly related to muscle loss and slower recovery from trauma, it is desirable to modulate the inflammatory response. As a result, benefits are believed to be derived from the administration of PKR-I to limit excess production of the cells that produce inflammation-modulating proteins (e.g., acute phase proteins (CRP), interleukins (IL-6, IL-1s, etc)).

Autoimmunity in a balanced and controlled manner is necessary for immuno-surveillance of potential neoplastic cells. Although autoimmunity has some side effects, it is still an important and natural process. Therefore, preventing the over-production of immune cells involved in manufacturing proteinaceous cytokines is expected to limit the immune response and prevent auto-immune disease.

For example, in systemic lupus erythematosus (SLE), PKR is overexpressed in activated SLE T cells, correlating with an increase in eIF2α phosphorylation. A high expression of PKR and subsequent eIF2α phosphorylation may be likely responsible, at least in part, for impaired translational and proliferative responses to mitogens in T cells from SLE patients. Grolleau, A. et al., J. Clin. Invest., 106(12):1561-1568, 2000.

In allergy—Allergic reactions to various antigens or effectors are mediated by the proteinaceous immunoglobulin E (IgE), which is produced by the B-cells when they come into contact with an antigen (e.g. pollen). The use of PKR-I is, therefore, believed to benefit the person by reducing the production of the IgE through limiting B-cells. This is similar to the function provided by a different type of compound called Omalizumab (Xolair®-Novartis). As opposed to a protein breakdown inhibitor, Omalizumab is a monoclonal antibody used in allergy-related asthma therapy, with the purpose of reducing allergic hypersensitivity.

In chronic obstructive pulmonary disease (COPD)—Because the conditions associated with COPD, including “chronic bronchitis,” are often related to hyperplasia and hypertrophy of mucous producing goblet cells of the airway, the use of PKR-I is believed to reduce symptoms associated with COPD. A reduction in secretion of mucus, which contributes to the airway obstruction, would therefore benefit a person treated with PKR-I. In addition, COPD is associated with inflammation, followed by scarring and remodeling of the tissue which narrows the airway.

In topical applications—PKR-I is believed to be of benefit for a variety of skin conditions which include, but may not be limited to, atopic dermatitis, eczema and psoriasis. In atopic dermatitis, there is an excessive reaction by the immune system producing inflamed, irritated and sore skin which may be controlled by administration of PKR-I.

In immunonutrition—The use of immunonutrition, such as Second Generation Impact® to modulate inflammation and reduce infection is common in patients undergoing surgery for cancer. Further control of cytokine production by PKR-I is expected to reduce the over-expression of inflammatory cytokines.

In chemotherapy—Recent research has suggested that specific bioactive peptides isolated from milk have cyto-protective properties when the cell cycle is arrested in the G₀ phase (cellular senescence). PKR-I is believed to alter the degradation of proteins that control the cell cycle. As a result, limiting cellular replication during active chemo- and radiotherapy is believed to protect healthy cells.

In diabetes—For Type I, as in autoimmunity, as described above. In Type II, beta cells of the pancreas secrete excess insulin prior to the final stages of full insulin-dependent, adult-onset diabetes. As a result, it is believed that PKR-I may be useful in a localized administration to prevent the hypersecretion of insulin.

In Cushing Disease—Cushing's Disease is caused by the presence of a tumor in the pituitary gland which promotes the secretion of excessive cortisol. As a result, it is believed that local and/or systemic administration of PKR-I will prevent tumor growth and likely reduce the synthesis of cortisol. Additional benefits may also be seen in other chronic stress responses where cortisol promotes lean body mass loss.

In organ transplantation—The use of PKR-I to reduce the expression of immune proteins that react to foreign antigen presentations thus precipitating organ rejection.

In rheumatic fever and rheumatic organ diseases—Rheumatic fever and rheumatic organ diseases are a inflammatory diseases that can develop as a rare complication of untreated or under treated strep infection, which is caused by infection with group A Streptococcus. The exact cause of rheumatic fever and rheumatic organ diseases is unknown but medical research has focused on an abnormal immune system response to the antigens produces by specific types of Streptococcal bacteria. The antigenic response from the infection results in a production of antibodies that attack organs, muscles and joints in error. There is no cure for rheumatic fever and rheumatic organ diseases but medical treatment for this condition involves antibiotic prescription to treat the streptococcal infection and prevent future infection and other medications ease the symptoms of the disease. Thus, temporarily diminishing the production of antibodies to provide time for antibiotic therapy would be accomplished by inhibition of protein synthesis via PKR-I.

In progeria—Progeria (Greek, “old age”), refers specifically to Hutchinson-Gilford Progeria syndrome or generally other accelerated aging diseases. Progeria is an extremely rare disease in which some aspects of aging are generally accelerated, with few affected children living past age 13. It is a genetic condition but occurs sporadically and is not inherited in families. There is no known cure for progeria but several discoveries have been made. Treatment with growth hormone (Sadeghi-Nejad, A. et al., J. Pediatr. Endocrinol. Metab., 20(5):633-637, 2007) and farnesyltransferase inhibitors (Meta, M. et al., Trends Mol. Med., 12(10):480-487, 2006) have been proposed. In 2003, M. Eriksson et al. reported that progeria may be a de novo dominant trait and develops during cell division in a newly conceived child or in gametes of one of the parents. It is caused by mutations in a LMNA (Lamin A) gene on chromosome 1. In progeria, the recognition site that the enzyme (protease) requires for cleaving Prelamin A to Lamin A is mutated. Lamin A cannot be produced and Prelamin A accumulates up on the nuclear membrane, causing a characteristic nuclear blebbing (Lans, H. et al., Nature, 440(7080):32-34, 2006). This results in the premature aging symptoms of progeria. A study which compared progeria patient cells with the skin cells from LMNA young and elderly human subjects found similar defects in the progeria and elderly cells, including down-regulation of certain nuclear proteins, increased DNA damage and demethylation of histone leading to reduced heterochromatin. The use of PKR-Inhibitor to reduce expression of proteins, such as proteins involved in nuclear lamina dysfunction (e.g., Prelamin A) that are reported to result in premature ageing.

The term “amino acids” as used herein, unless otherwise stated, refers to at least one of essential amino acids, e.g. isoleucine, leucine, lysine, methionine, phenylalanine, threonine, tryptophan, valine, or histidine; conditionally essential amino acids, e.g., tyrosine, cysteine, arginine, or glutamine; or non-essential amino acids, e.g. glycine, alanine, proline, serine, glutamic acid, aspartic acid, asparagines, taurine or carnitine.

The term “essential amino acids” (EAA) as used herein, unless otherwise stated, refers to at least a source of one of the amino acids: isoleucine, leucine, lysine, methionine, phenylalanine, threonine, tryptophan, valine, and histidine.

In addition, the amino acids arginine, cysteine, glycine, glutamine and tyrosine are considered conditionally essential, meaning they are not normally required in the diet, but must be supplied exogenously to specific populations that do not synthesize it in adequate amounts.

As used herein, “branched-chain amino acid” refers to at least one of the amino acids, e.g., leucine, isoleucine or valine.

L-Theanine or gamma-ethylamino-L-glutamic acid (also known as Suntheanine™) is a unique amino acid found only in tea leaves, e.g., black, oolong, and green tea (infusions of Camellia sinensis). Theanine is related to glutamine, and can cross the blood-brain barrier. Because it can enter the brain, theanine has psychoactive properties. Theanine has been shown to reduce mental and physical stress and may produce feelings of relaxation and improves cognition and mood when taken in combination with caffeine. L-theanine as been shown to promote the generation of alpha-brain waves, an index of relaxation. It may also boost natural resistance to microbial infections and perhaps even tumors. A dose of 50 to 200 mg may provide a relaxation effect. No dosage of L-theanine is suggested for enhanced immune system functioning; however, volunteers in a pilot study consumed approximately 600 mL of tea a day.

As used herein, “prebiotics” are non-digestable food ingredients that, when provided to the digestive tract of the host or subject, selectively stimulate the growth and/or activity of one or a limited number of beneficial bacterial species over the pathogenic ones. Prebiotics include yeast, yeast cultures, fungal cultures, and known dietary fibers such as polysaccharides and oligosaccharides such as fructooligosaccharides (FOS) and guar gums, especially partially hydrolysedguarm gum (PHGG) and pectins. “Probiotics” are actual bacterial species, that when introduced to the digestive tract of the host or subject, actually colonize and produce beneficial effects. Preferably, the probiotics include one or more of a Lactobacilli and Bifidobacteria. The term “synbiotics” refers to mixtures of prebiotics and probiotics that beneficially affect the host by improving the survival and implantation of live microbial dietary supplements in the gastrointestinal tract of the host or subject.

“Essential fatty acids” or “EFA” may refer to any fatty acids that may be used by the body and may be classified as either saturated, polyunsaturated or monounsaturated fatty acids that may be found in nature or produced synthetically. EFA may include, without limitation, cholesterol, prostaglandins, lecithin, choline, inositol, conjugated linolenic acid, myristic acid, palmitic acid, stearic acid, oleic acid, α-linolenic acid, eicosapentaenoic acid, docosapentaenoic acid, docosahexanoic acid, linolenic acid, γ-linolenic acid, ω-3 fatty acids, ω-6 fatty acids, ω-9 fatty acids, polyunsaturated fatty acids, long-chained polyunsaturated fatty acids, arachidonic acids, monounsaturated fatty acids, precursors of fatty acids and derivatives of fatty acids. A useful composition for preventing or treating cachexia or anorexia according to the present invention may include a combination of a mixture of at least one of the EFAs.

The daily delivery of the nutrients referred hereinabove may vary depending on body weight, sex, age, and/or medical condition of the individual or subject.

### Nutritional Interventions

The use of targeted nutrients to increase cytotoxicity during active radio- and/or chemotherapy treatments or use in combination with PKR-I according to the present invention to achieve the desired effects are as follows:

In cytotoxicity: Free radical inducing nutrients are believed to increase damage to diseased and tumor cells. Examples include the following:
a. Iron, nitrites/nitrates
b. Low status of vitamins E, C, B-complex, and selenium and other anti-oxidants
c. Elevated phytate (divalent chelator), L-theanine to block glutamine uptake

In cyto-protection, the following nutrients are believed to prevent normal cells from immune attack and cell damage and injury during chemotherapy:
a. Antioxidants: glutamine, cysteine, Vitamins A, C, E, and Selenium
b. Lysine→Norleucine (a BCAA analog) for anabolism
c. Nucleotides (or fragments of nucleotides in circulation) for anabolism
d. High polyunsaturated fatty acid (PUFA)/monounsaturated fatty acid (MUFA) to increase membrane fluidity and prevent reactive oxidant damage
e. Sialic acid oligosaccharides to improve gut cell health and reduce infiltration of pathogenic organisms and compounds.

The uses of products such as probiotics, prebiotics, and synbiotics are well documented. For example, probiotics are known to (a) lower the frequency and duration of diarrhea associated with antibiotics, rotavirus infection, chemotherapy, and to a lesser extent, traveler's diarrhea; (b) stimulate cellular and humoral immunity; and (c) decrease unfavorable metabolites such as ammonium and procarcinogenic enzymes in the colon. Probiotics has a possible role in cancer prevention. See Schrezenmeir, J. et al., Am. J. Clin. Nutr., 73(Suppl.):3615-3645, 2001. As used herein, the term “probiotics” refers to a preparation of or a product containing viable defined microorganism in sufficient numbers, which alter the microflora (by implantation or colonization) in a compartment of the host and by that exert beneficial effects in this host. The term “prebiotics” refers to a non-digestible food ingredient that beneficially affects the host by selectively stimulate the growth and/or activity of one or a limited number of bacteria in the colon. For example, a bifidobacteria would be promoted by ingestion of substances such as fructooligosaccharides, inulin, transgalactosylated oligosaccharides, and soybean oligosaccharides. The term “synbiotic” is used when a product contains probiotics and prebiotics. Since the term “synbiotic” alludes to synergism, it is reserved for products in which a prebiotic compound selectively favors the probiotic compound. In strict sense, it is a product that contains an oligofructose and a probiotic bifidogenic bacteria. See Schrezenmeir, J. et al., 2001 supra.

The use of a kinase-inhibitor with or without nutritional compounds potentiates chemotherapeutic agent efficacy in treatment of cancerous tumors. Additional benefits include the nutritional modulation of metabolic pathway that regulates muscle loss, specifically cancer cachexia.

Upon the administration of PKR-I, the phosphorylation of double-stranded RNA Protein Kinase resulted to a reduction of the growth of cells (MAC16 solid tumor) more effectively in combination with chemotherapy agent (e.g., 5-fluorouracil or gemcitabine) than when either was used alone.

Additionally, administration of a PKR-inhibitor may be used to reduce the active form of the proinflammatory cytokine Nuclear Factor-kappa-B (NF-kB). NF-kB is thought to be related to the resistance by certain tumor cells to chemotherapy drugs, for example gemcitabine (Arlt, A. et al., Oncogene, 22(21):3243-3251, 2003) and 5-FU (Uetsuka, H. et al., Exp Cell Res., 289(1):27-35, 2003). As a result, the administration of PKR-inhibitor may be direct or indirect in its ability to potentiate chemotherapy. Both of which are compounds commonly used in the treatment of neoplastic growths (e.g. colon cancer).

The inventors have shown that the PKR-inhibitors reduce the growth of cancer cells when introduced at very specific concentrations (maximal effect at 200 nM, diminished effect at lower and greater concentrations). In addition, the inhibition of PKR further decreased the proliferation of cancer cells exposed to chemotherapy drugs. The cellular inhibition appears to have been a synergistic effect as compared to the inhibition observed with either compound alone (see FIG. 8). The administration of specific nutritional compounds, structurally unrelated to the PKR-inhibitor compounds, is believed to also reduce cancer cell growth and prevent cancer cachexia. However, the nutritional compounds act through a different mechanism than the PKR-inhibitor(s) compounds previously described (Jammi et al. 2003).

### Nutritional Alleviation of Immune Suppression by Chemotherapy:

Some chemotherapeutic agents are known to cause immune depression, two examples include 5-Fluorouracil and gemcitabine.

5-fluorouracil (5-FU) is a common chemotherapy drug that is given as a treatment for some types of cancer, including: bowel, breast, stomach, and esophageal cancer. A complication associated with the use of 5-FU is lowered resistance to infection. 5-FU can reduce the production of white blood cells by the bone marrow, making the patient more prone to infection.

Gemcitabine is a chemotherapy drug that is given as a treatment for non-small cell lung cancer, pancreatic, bladder, and breast cancer. Gemcitabine can also reduce the production of white blood cells by the bone marrow, increasing the patient's susceptibility to infection. Significant reduction in immune function typically begins seven days after treatment dosing and resistance to infection is typically lowest between 10-14 days after chemotherapy. Blood cells will often increase steadily, and return to normal levels before your next course of chemotherapy is due.

Glutamine is believed to provide benefits to patients receiving chemotherapy by supporting immune function. Nutrient interaction with chemotherapy has been previously suggested. Antioxidants decrease the efficacy of chemotherapy by prematurely breaking down the drug within cells, which is beneficial to healthy cells, but undesirable in tumor cells. The amino acid glutamine may promote chemotherapeutic drug breakdown because it is a component of the intercellular antioxidant glutathione (GSH) (Rouse, K. et al., Annals Surge., 221(4):420-426, 1995). Therefore, blocking glutamine uptake by the cell with the amino acids L-theanine has been suggested as a method to potentiate chemotherapy in the case of doxorubicin (Sugiyama, T. and Adzuki, Y., Biochip. Biophys. Act, 1653(2):47-59, 2003). However, not all research supports this claim against glutamine (Rubio, I. T. et al., Ann Surg., 227(5):772-778, 1998). GSH may promote degradation of chemotherapeutic compounds in an effort to protect the cell, but glutamine is also a vital component of proper immune cell function. Despite evidence that would suggest reducing glutamine, the effect of immune function would compromise patient health and recovery.

Immunonutrition: The ostentation of chemotherapeutic efficacy (with PKR-inhibitors) is likely to increase the risk of infection to the patient. Infection and risk of infection may reduce the oncologists willingness to administer aggressive doses of chemotherapy necessary for successful treatment. In addition, infection may also compromise the patient's ability to tolerate a potent treatment regimen as well as recover/heal from treatment related comorbidities or surgical wounds. Nutritional supplementation with ingredients including anti-inflammatory fatty acids (e.g., eicosapentanoic acids and docosahexanoic acid), the amino acids L-arginine and its precursor, L-citrulline, and the ribonucleic acids can promote immune health through T-cell activation, maturation and reduced inflammation.

Bioactive milk-derived proteins: Bioactive milk-derived proteins provide a source of bioactive peptides (e.g., transforming growth factor-beta (isoforms 1-3) which slow or temporarily arrest cell cycle division in healthy cells. These bioactive proteins require activation through processing, such as acidification and thus standard milk is not suitable. Administration of these bioactive proteins from milk is believed to protect cells which come into contact with the protein, such as the oral, esophagel and gastrointestinal epithelium. The bioactive peptides work by decreasing the susceptibility of rapidly dividing cells to damage by chemotherapeutic agents (with or without PKR-inhibitors).

Nucleotides (e.g., ribonucleic acids): The compounds (e.g., adenine, guanine, cytosine) are believed to provide immune system support to a cancer patient receiving chemotherapy, especially if the chemotherapy regimen is provided in combination with a potentiator, such as a PKR-inhibitor(s). Nucleotides support bone marrow creation and its product which include both the red blood cells and T-cell (immune cell) maturation. In addition, nucleotides are being investigated for their potential to promote drug absorption, therefore the nutritional supplementation of nucleotides is believed to be a benefit by increase chemotherapy agent uptake by tumor cells and support immune function.

Angiogenic and vasodilatory nutrients: Nutrients which promote angiogenesis and blood flow also increase delivery of chemotherapeutic agents to metabolically active tissue. The nutritional administration of amino acids L-arginine and/or L-citrulline, as well as the catechin flavanol compounds (which are considered as cancer chemopreventive agents), are not recommended in cancer patients as they may promote angiogenesis, a property of invasive tumors with rapid growth. However, increased blood delivery specifically to the tumor would also increase the uptake of cytotoxic chemotherapeutic agents.

Administration of PKR-Inhibitors with or without Specific Nutrients on the Attenuation of Cancer Cachexia.

The loss of muscle protein in cancer cachexia, cardiac cachexia, and possibly other conditions including sarcopenia, HIV/AIDS, etc is controlled by the subunit association and/or upregulated proteosome production. One of the steps in this process involves activation of the eukaryotic initiation factor 2-alpha (eIF2α). The activation, via phosphorylation, of eIF2αpromotes proteosomal protein degradation. Administration of PKR-inhibitors decreases activation (phosphorylation) of the eIF2α molecule thus reducing activation of the proteosome and reducing muscle protein breakdown.

Increasing the potency of chemotherapy and inhibiting the process of cancer cachexia via administration of PKR-inhibitors, with or without additional nutrients has been shown to increase the effect of chemotherapy drugs on tumor cells. The benefit of this invention is that it may reduce the length of time or number of doses necessary to elicit a clinically-relevant effect. Research has previously been conducted to evaluate compounds that inhibit-PKR, but it has not been previously demonstrated that inhibition of PKR would have cancer treatment benefits. Additional benefits may also be obtained with the administration of PKR-inhibitors and specific nutritional compounds, including amino acids, fatty acids, nucleic acids, to further potentiate chemotherapy and attenuate therapy associated toxicities.

PKR-inhibitors block the phosphorylation of PKR involved in cancer cachexia and tumor resistance to therapy. Our results demonstrate the exciting potential of these compounds in cancer therapy. In addition, the use of specific nutrients, which act upon a separate protein (PPI) or PKR directly, to reduce phosphorylation of PKR also suggests they have potential as co-therapeutic agents in cancer treatment. The benefits of the nutrients (amino acids, polyphenolics) as compared to the pharmaceutical grade compounds are: alternate mechanism of action, price, safety, and availability via alternative retail channels.

As used herein the meaning of the terms “active agent”, “active ingredient”, “active compound” or in some cases “compound” is to be understood as equivalent.

It follows that the terms “biological activity of PKR” and “biologically active PKR” refer to any biological activity associated with PKR, or a fragment, derivative, or analog of PKR, such as enzymatic activity, specifically including autophosphorylation activity and kinase activity involving phosphorylation of substrates such as eukaryotic translation initiation factor 2 (eIF-2) and transcription factors such as NF-κB.

By “ex vivo” is meant outside the body of the organism from which a cell or cells is obtained or from which a cell lineage is isolated. Ex vivo applications may comprise use of intact cells, or employ a cell-free system (i.e., in vitro) such as a lysate.

By “in vivo” is meant within the body of the organism from which the cell was obtained or from which a cell lineage is isolated.

By “human cell” is meant a cell isolated from humans at any stage of development.

By “patient or subject” is meant any animal.

Animals include, but is not limited to avians and mammals which includes but is not limited to rodents (murine), aquatic mammals, domestic animals such as canines, lupines, rabbits and felines, farm animals such as sheep (ovine), pigs (porcine), cows (bovines), goats (hircrine) and horses (equine), and humans. Wherein the terms animal or mammal or their plurals are used, it is contemplated that it also applies to any animals that are capable of the effect exhibited or intended to be exhibited by the context of the passage. Other animals that can be treated using the methods, compositions, and kits of the invention include lizards, snakes, fish, and birds.

By “mammal” is meant to include but is not limited to: rodents (murine), aquatic mammals, domestic animals such as canines, lupines, rabbits and felines, farm animals such as sheep (ovine), pigs (porcine), cows (bovines), goats (hircrine) and horses (equine), and humans. Wherein the term mammal is used, it is contemplated that it also applies to other animals that are capable of the effect exhibited or intended to be exhibited by the mammal.

The term “residue” or “amino acid residue” or “amino acid” as used herein refers to an amino acid that is incorporated into a protein, polypeptide, or peptide (collectively “peptide”). The amino acid may be a naturally occurring amino acid and, unless otherwise limited, may encompass known analogs of natural amino acids that can function in a similar manner as naturally occurring amino acids.

The term “cancer” refers to various types of malignant neoplasms, most of which can invade surrounding tissues, and may metastasize to different sites (PDR Medical Dictionary 1st edition (1995)).

The terms “neoplasm” and “tumor” refer to an abnormal tissue that grows by cellular proliferation more rapidly than normal and continues to grow after the stimuli that initiated proliferation is removed (PDR Medical Dictionary 1st edition (1995)). Such abnormal tissue shows partial or complete lack of structural organization and functional coordination with the normal tissue which may be either benign (i.e., benign tumor) or malignant (i.e., malignant tumor).

The language “treating a disorder associated with aberrant cellular proliferation” is intended to include the prevention of the growth of neoplasms in a subject or a reduction in the growth of pre-existing neoplasms in a subject. The inhibition also can be the inhibition of the metastasis of a neoplasm from one site to another. In one aspect, the neoplasms are sensitive to one or more translation initiation inhibitors described herein. Examples of the types of neoplasms intended to be encompassed by the present invention include but are not limited to those neoplasms associated with cancers of the breast, skin, bone, including bone marrow and hemopoietic tissues, prostate, ovaries, uterus, cervix, liver, lung, brain, larynx, gallbladder, pancreas, rectum, parathyroid, thyroid, adrenal gland, immune system, neural tissue, head and neck, colon, stomach, bronchi, and/or kidneys.

As used herein, a “test sample” refers to a biological sample obtained from a subject of interest. For example, a test sample can be a biological fluid sample (e.g., serum, sputum, urine), tissue sample (e.g., a biopsy) or cell sample (e.g., a cheek scraping). As used herein, a “normal sample” or a “standard sample” refers to a biological sample obtained from a healthy (i.e., non-malignant) biological fluid sample, tissue sample or cell sample. As used herein, the term “biological sample” is intended to include tissues, cells and biological fluids isolated from a subject, as well as tissues, cells and fluids present within a subject. Biological samples may be of any biological tissue or fluid or cells. Typical biological samples include, but are not limited to, sputum, lymph, blood, blood cells (e.g., white cells), fat cells, cervical cells, cheek cells, throat cells, mammary cells, muscle cells, skin cells, liver cells, spinal cells, bone marrow cells, tissue (e.g., muscle tissue, cervical tissue, skin tissue, spinal tissue, liver tissue and the like) fine needle biopsy samples, urine, cerebrospinal fluid, peritoneal fluid and pleural fluid, or cells therefrom. Biological samples may also include sections of tissues such as frozen sections taken for histological purposes. A biological sample may be obtained from a mammal, including, but not limited to horses, cows, sheep, pigs, goats, rabbits, guinea pigs, rats, mice, gerbils, non-human primates and humans. Biological samples may also include cells from microorganisms (e.g., bacterial cells, viral cells, yeast cells and the like) and portions thereof.

MAC16 tumor is derived from an established series (MAC) of chemically induced, transplantable colon adenocarcinomas and is being produced by a particular cell line now deposited on 8 Mar. 1989 in the European Collection of Animal Cell Cultures (ECACC) at the Public Health Laboratory Service Centre for Applied Microbiology and Research, Porton Down, Salisbury, Wiltshire, United Kingdom under a provisional accession number 8903016.

The MAC16 tumor is a moderately well-differentiated adenocarcinoma, which has been serially-passaged in mice for many years. It has been found that it appears to represent a more satisfactory experimental model for tumors which induce cachexia in human patients, especially insofar as it has often been found to produce substantial loss of body weight at small tumor burdens (less than 1% body weight) and without a reduction in the intake of either food or water.

### Pharmaceutical Compositions

The compounds or agents of the present invention described herein are compounds or agents that affect eIF2α or PKR phosphorylation or potentiate the compounds or agents that affect eIF2α or PKR phosphorylation, for example, by inhibition of eIF2α or PKR phosphorylation. The compounds or agents of the present invention can be incorporated into pharmaceutical compositions suitable for administration.

The compound of the present invention that includes at least one inhibitor of PKR-I, at least one phosphorylation inhibitor of PKR-I and/or potentiator of PKR-I may be administered enterally or parent rally. The parenteral administration may be selected from a group consisting of subcutaneous, intravenous, intramuscular, and topical administration. The enterable administration may be in form of a tablet, liquid, gel, sachet, powder, lozenge, film, gum, and capsule. In addition, the route of enterable administration method may be selected from a group consisting of intranasal, interiorly, nasogastric, orogastric, gastric port, jejunal port, and ileal port.

The compound of the present invention may typically comprise the above-mentioned compound(s) and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active agent, use thereof in the compositions is contemplated. Supplementary nutritional agents can also be incorporated into the compositions of the present invention.

A pharmaceutical composition of the invention is formulated to be compatible with its intended route of administration. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active agent in the required amount in an appropriate solvent with one or a combination of ingredients enumerated above, as required, followed by filtered sterilization. Generally, dispersions are prepared by incorporating the active agent into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active agent can be incorporated with excipients and used in the form of tablets, troches, or capsules. Oral compositions can also be prepared using a fluid carrier for use as a mouthwash, wherein the agent in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or agents of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In one embodiment, the compounds of the present invention are prepared with carriers that will protect the agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811, incorporated herein by reference in its entirety.

### Nutritional Compositions

Chemotherapy and radiotherapy not only are effective in destroying cancer cells but they also are harmful to non-cancer cells by causing premature death of these cells. In addition, the compounds of the present invention not only can inhibit the growth of tumor cells by inhibiting the phosphorylation of PKR but can also enhance the efficacy of chemotherapeutic agents in cancer patients. As discussed hereinabove, the compounds of the present invention include at least one PKRI either alone or in combination with at least one potentiator. Beside being formulated for pharmaceutical purposes, these compounds can be nutritionally formulated to achieve the desired purposes, as discussed hereinabove.

A nutritional composition according to the present invention may be in form of a dietary means, e.g., supplements or in form of a nutritional formulation, e.g., medical food or beverage product, e.g., in form of a complete meal, part of a meal, as food additive or as powder for dissolution. The powder may be combined with the liquid, e.g., water or other liquid, such as milk or fruit juice.

Optionally, the nutritional formulation, not only includes the compounds of the present invention, may be nutritionally complete, i.e., may include minerals, vitamins, trace elements, and fat and/or fatty acid sources so that they may be used as the sole source of nutrition supplying essentially all the required daily amounts of vitamins, minerals, carbohydrates, fat and/or fatty acids, proteins and the like.

Accordingly, the nutritional compositions of the present invention may be provided in the form of a nutritionally balanced complete meal, e.g. suited for oral or tube feeding, e.g. by means of nasogastric, nasoduodenal, esophagostomy, gastrostomy, or jejunostomy tubes, or peripheral or total parenteral nutrition. Preferably, the compositions of the invention are for oral administration.

The nutritional compositions of the present invention may be useful for promoting muscle protein synthesis or controlling tumor-induced weight loss, such as cachexia, e.g. cancer cachexia. It may also be useful as nutritional supplement for patients suffering from an autoimmune disease or other disorder for which chemotherapeutic agents are used.

In one feature of the invention, the nutritional composition may further include but not limited to a bioactive protein, a branched-chain amino acid, an essential amino acid, an amino acid or amino acid analog, a nucleotide or RNA, a vitamin, a glutamine, a sialic acid oligosaccharide, an L-theanine, a prebiotic, a probiotic or a synbiotic, an essential fatty acid, a PUFA and/or MUFA, a dietary oil and an anti-oxidant.

Dietary oils may be used in the preparation of the nutritional compositions of the invention. Dietary oils include but are not limited to canola, medium chain triglycerides (MCT), fish, soybean, soy lecithin, corn, safflower, sunflower, high-oleic sunflower, high-oleic safflower, olive, borage, black currant, evening primrose and flaxseed oil.

The nutritional composition of the present invention may further include soluble fibers, e.g. agar, alginates, carubin, pectin and its derivatives, e.g. pectins from fruits and vegetables, and more preferably pectins from citrus fruits and apple, beta-glucan, such as oat beta-glucan, carrageenans, e.g. kappa, lambda and iota carrageenans, furcellaran, inulin, arabinogalactan, cellulose and its derivatives, scleroglucan, psyllium, such as psyllium seed husk, mucilages and gums. According to the invention, gums and mucilages are preferably plant exudates. In particular, the term “gum” as used herein refers to the commonly available vegetable gums and more particularly to konjac gum, xanthan gum, guar gum (guaran gum), locust bean gum, tara bean gum, gum tragacanth, arabic gum, karaya gum, gum ghatti, gellan gum and other related sterculia gum, alfalfa, clover, fenugreek, tamarind flour. Native and modified, e.g. hydrolyzed, soluble fibers may be used according to the invention. According to the invention, preferably guar gum, e.g. hydrolyzed guar gum, may be used.

The daily delivery of the optional nutrients referred to hereinabove may vary depending on body weight, sex, age and/or medical condition of the individual.

The nutritional composition of the invention may include one or more fatty acids, for example, polyunsaturated fatty acids, prebiotics or probiotics or a combination of prebiotics and probiotics (synbiotics); and bioactive compounds or extracts.

The nutritional composition may provides at least 100%, e.g. 100%, of the U.S. RDA for vitamins and minerals per daily dose, e.g., calcium, magnesium, iron, zinc, phosphorus, vitamin D, vitamin K. It may also contain anti-oxidants including, but not limited to, glutamine, cysteine, vitamins A, C, E and selenium. It may particularly contains high amounts of vitamin E, which is useful in the compositions for promotion of muscle protein synthesis or controlling tumor-induced weight loss, such as cachexia, e.g. cancer cachexia.

Nutritional compositions in accordance with the present invention may be provided as a medical food or beverage product, e.g. in oral nutritional form, e.g. as a health drink, as a ready-made drink, optionally as a soft drink, including juices, milk-shake, yogurt drink, smoothie or soy-based drink, in a bar, or dispersed in foods of any sort, such as baked products, cereal bars, dairy bars, snack-foods, soups, breakfast cereals, muesli, candies, tabs, cookies, biscuits, crackers (such as a rice crackers), and dairy products.

Preferably, the compositions of the invention may be administered as a nutritional formulation, e.g. as part of a meal, e.g. in the form of a health drink, e.g. ready-to-use drink.

Solid oral dosage forms are prepared in a manner known per se, for example by means of conventional mixing, granulating, confectioning, dissolving or lyophilizing processes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

### EXAMPLES

The present disclosure is further defined in the following Examples. It should be understood that these Examples, while indicating preferred embodiments, are given by way of illustration only. From the above discussion and these Examples, one skilled in the art can ascertain the preferred features of this disclosure, and without departing from the spirit and scope thereof, can make various changes and modifications to adapt it to various uses and conditions.

### Materials:

Fetal calf serum (FCS) and RPMI 1640 tissue culture medium were purchased from Invitrogen (Paisley, Scotland). L-[2,6-³H] phenylalanine (spec. act., 2.00TBqmmol⁻¹), Hybond A nitrocellulose membranes and enhanced chemiluminescene (ECL) development kits were from Amersham Biosciences (Bucks, UK). Rabbit monoclonal antibodies to phospho and total PKR were purchased from New England Biolabs (Herts, UK). Rabbit polyclonal antisera to phospho eIF2α was from Abcam (Cambridge, UK) and to total eIF2α from Santa Cruz Biotechnology (CA). Peroxidase-conjugated goat anti-rabbit antibody was purchased from Dako Ltd (Cambridge, UK). The PKR Inhibitor and PhosphoSafe™ Extraction Reagent were from Merck Eurolab Ltd (Leics, UK). EMSA (Electrophoretic Mobility Shift Assay) gel shift assay kits were from Panomics (CA, USA). Gemcitabine (Gemzar®) was a gift from Eli Lilly and Co (Basingstoke, UK). 5-fluorouracil was purchased from Sigma Aldridge (Dorset, UK).

### Maintenance of Tumors

MAC16, which was first described by Cowen et al. (J. Natl. Cancer Inst., 64:675-681, 1980), are pure NMRI mouse strain bearing an established series (MAC) of chemically-induced transplantable colon adenocarcinomas. The MAC16 tumor is a moderately well-differentiated adenocarcinoma, which has been serially passaged in mice for many years. It represents a more satisfactory experimental model for tumors which induce cachexia in human patients, especially insofar as it has often been found to produce substantial loss of body weight at small tumor burdens (less than 1% body weight) and without a reduction in the intake of either food or water (Bibby, M. C. et al., J. Natl. Cancer Inst., 78:539-546, 1987).

The MAC16 and MAC13 tumors were propagated in vitro in RPMI 1640 medium containing 10% FCS at 37° C., under an atmosphere of 5% CO₂ in air. For cell growth assays, cells were seeded at either 0.5×10⁵ cells per well (MAC13) or 1×10⁵ cells per well (MAC16) in 24 well multi-well dishes and allowed to accumulate for 24 h prior to drug addition. Cell number was determined three days later, whilst the cells were in exponential growth.

Both the MAC16 and MAC13 tumors were passaged in vivo in NMRI mice by transplanting fragments subcutaneously (s.c.) into the flank, as described in Bibby et al., J. Natl. Cancer Inst., 78(3):539-546, 1987. To maintain cachexia, the MAC16 tumor for passage was selected from donor animals with established weight loss, and treatment was initiated when the average weight loss was 5%. Animals were randomized into groups of six to receive solvent (DMSO (dimethylsulfoxide): PBS (phosphate-buffered saline); 1:20) or the PKR inhibitor at 1 and 5 mg/kg administered daily by s.c. injection. Animals were terminated by cervical dislocation when the body weight loss reached 20%. All animal experiments followed a strict protocol approved by the British Home Office, and the ethical guidelines that were followed meet the standards required by the UKCCR guidelines (Workman, P. et al., Br. J. Cancer, 77:1-10, 1998).

### Measurement of Protein Synthesis

Protein synthesis in MAC16 and MAC13 cells was determined by the incorporation of L-[2,6-³H] phenylalanine into protein over a 4-hour period, as described in Eley, H. L. and Tisdale, M. J., J. Biol. Chem., 282(10):7087-7097, 2007. The reaction was terminated by removal of tissue culture medium, and washing three times with ice-cold sterile PBS. The PBS was removed and ice-cold 0.2M perchloric acid was added, followed by incubation for 20 min at 4° C. Following removal of perchloric acid, 0.3M NaOH was added, and incubation ensued for 30 min at 4° C. The reaction was proceeded by a further incubation for 20 min at 37° C., and 0.2M perchloric acid was added. The mixture was left on ice for another 20 min. Following centrifugation at 700 g for 5 min at 4° C., the protein-containing pellet was dissolved in 0.3 M NaOH, and the radioactivity was determined. The protein content was analyzed using a standard colorimetric protein assay (Sigma).

### Western Blot Analysis

Samples (approximately 10 mg) of tumor were homogenised in 500 μl of PhosphoSafe™ Extraction Reagent and centrifuged at 15,000 g for 15 min at 4° C. Portions of cytosolic protein (10 μg) were resolved on 10% sodium dodecyl sulphate/polyacrylamide gels (SDS/PAGE; 6% for eIF2α). The resolved proteins were transferred onto 0.45 μm nitrocellulose membranes, which had been blocked with 5% Marvel in Tris-buffered saline, pH 7.5, at 4° C. overnight. Membranes were then washed for 15 min in 0.5% Tween-buffered saline, or TBS-Tween, prior to the addition of primary antibodies. The primary antibodies were used at a dilution of 1:1000, except for phospho eIF2α, which was used at 1:500. The primary antibodies were washed off the membranes for 15 min, with buffer changes every 5 min, using 0.1% TBS-Tween. The secondary antibodies were used at a dilution of 1:1000, and were washed off after 45 min. Development was by ECL, and films were developed for 3-6 min. Blots were scanned by a densitometer to quantify differences.

### Electrophoretic Mobility Shift Assay (EMSA)

DNA-binding proteins were isolated from tumor samples by hypotonic lysis, followed by high salt extraction of nuclei according to the method of Andrews and Faller (Nucleic Acids Res., 19(9):2499, 1991). EMSA was carried out using a Panomics EMSA “gel shift” kit, according to the manufacturer's instructions

### Statistical Analysis

Results are presented at mean±SEM for at least three replicate experiments. Differences in means between groups were determined by one-way analysis of variance (ANOVA), followed by Tukey-Kramer multiple comparison test. P values less than 0.05 were considered significant.

### Results

In cancer patients weight loss is not only an independent predictor of a shorter survival time, but it also decreases response to treatment, as well as predicting toxicity from treatment (Ross, P. J. et al., Br. J. Cancer, 90(10):1905-1911, 2004). Weight loss is due to progressive atrophy of skeletal muscle and adipose tissue induced by cytokines and tumor factors, such as proteolysis-inducing factor (PIF) and lipid mobilising factor (LMF) (Tisdale, M. J., Curr. Opin. Clin. Nutr. Metab. Care, 5(4):401-405, 2002). Such factors may influence metabolism, not only in the host tissues, but also the primary tumor and metastases. Thus LMF induces expression of uncoupling protein (UCP) 2 in tumors, which is thought to be involved in the detoxification of free radicals, and this protects tumor cells from cytotoxic drugs generating free radical damage (Sanders, P. M. and Tisdale, M. J., Br. J. Cancer, 90(6):1274-1278, 2004). Expression of the PIF core peptide, dermicidin, in breast cancer cells promotes cell growth and survival and reduces serum dependency (Porter, D. et al., Proc. Natl. Acad. Sci. USA, 100:10931-10936, 2003). PIF has been shown to promote muscle atrophy through activation of the transcription factor nuclear factor-κB (NF-κB) by a mechanism involving activation, by autophosphorylation, of the dsRNA-dependent protein kinase PKR (Eley and Tisdale, 2007). A recent study (Eley et al, 2007) using a low molecular weight inhibitor of PKR in mice bearing the cachexia-inducing MAC16 tumor showed that it not only attenuated muscle atrophy, but also inhibited tumor growth. This was surprising, since like human tumors which induce cachexia, the MAC16 tumor is highly chemoresistant (Double, J. A. and Bibby, M. C., J. Natl. Cancer Inst., 81(13):988-994, 1989). This is the first report indicating that inhibition of PKR induced tumor growth inhibition and studies into the mechanism of this effect may provide an insight into the treatment of chemoresistant tumors.

One possible link between PKR and tumor growth involves activation of NF-κB. Activation of NF-κB has been connected with tumor cell survival and proliferation, as well as invasion and angiogenesis, critical events for tumor metastasis (Karin, M., Nature, 441(7092):431-436, 2006). NF-κB has been reported to be constitutively activated in a number of tumor types including colorectal carcinoma (Kojima, M. et al., Anticancer Res., 24(2B):675-681, 2004), pancreatic adenocarcinoma (Wang, W. et al., Clin. Cancer Res., 5:119-127, 1999) and hepatocellular carcinoma (Tai, D. I. et al., Cancer, 89:2274-2281, 2000). The factors responsible for constitutive activation of NF-κB include tumor necrosis factor-α (TNF-α), interleukin-1 (IL-1), pH and hypoxia (Baldwin, A. S., J. Clin. Invest., 107(3):241-243, 2001). It is possible that production of PIF by cachexia-inducing tumors may also lead to constitutive activation of NF-κB, as it does in the skeletal muscle of cachectic animals (Wyke, S. M. et al., Br. J. Cancer, 91(9):1742-1750, 2004). Inhibition of NF-κB activation in skeletal muscle by resveratrol also inhibited tumor growth in mice bearing the MAC16 tumor, although the mechanism was not investigated. NF-κB can activate the transcription of genes which suppress apoptosis, through the regulation of caspase activity (Karin, M. et al., Nat. Immunol., 3(3):221-227, 2002). Inhibition of apoptosis by NF-κB renders tumors resistant to chemotherapy and radiation (Bharti, A. C. and Aggarwal, B. B., Ann. NY Acad. Sci., 973:392-395, 2002), and could explain why cachexigenic tumors are so resistant to therapy.

This study compares the effectiveness of a PKR inhibitor on growth of the MAC16 tumor, with that on the MAC13 tumor, which is histologically similar to the MAC16 tumor, but does not induce cachexia (Beck, S. A. and Tisdale, M. J., Cancer Res., 47:5919-5923, 1987), and investigates the mechanism of tumor growth inhibition.

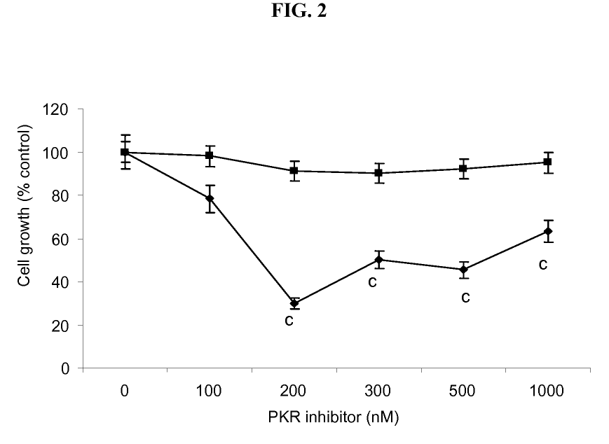

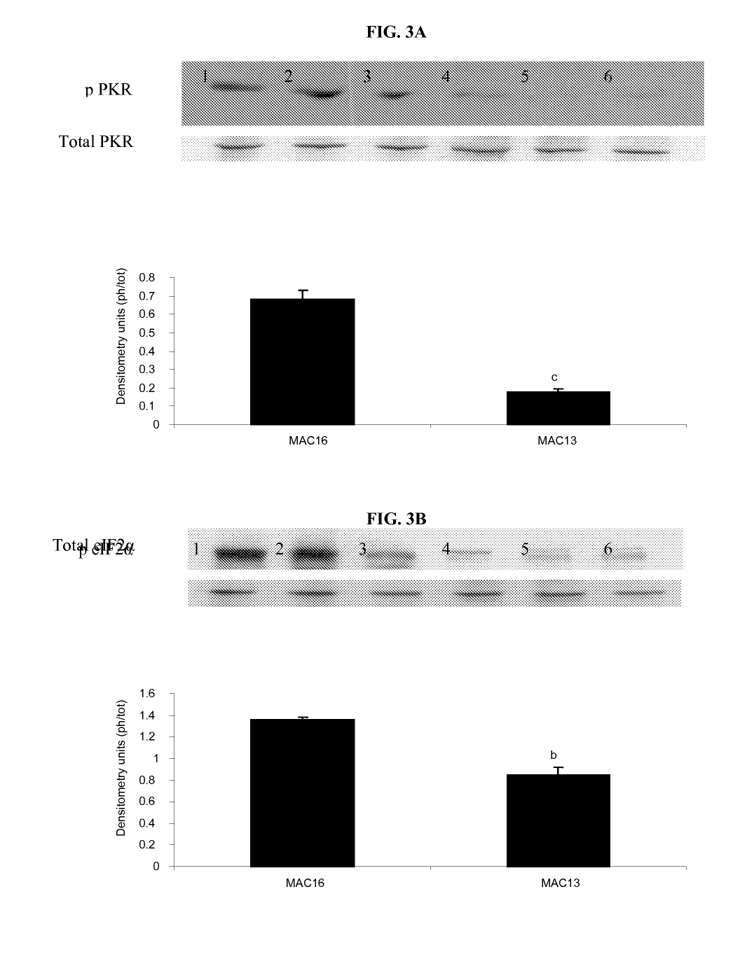

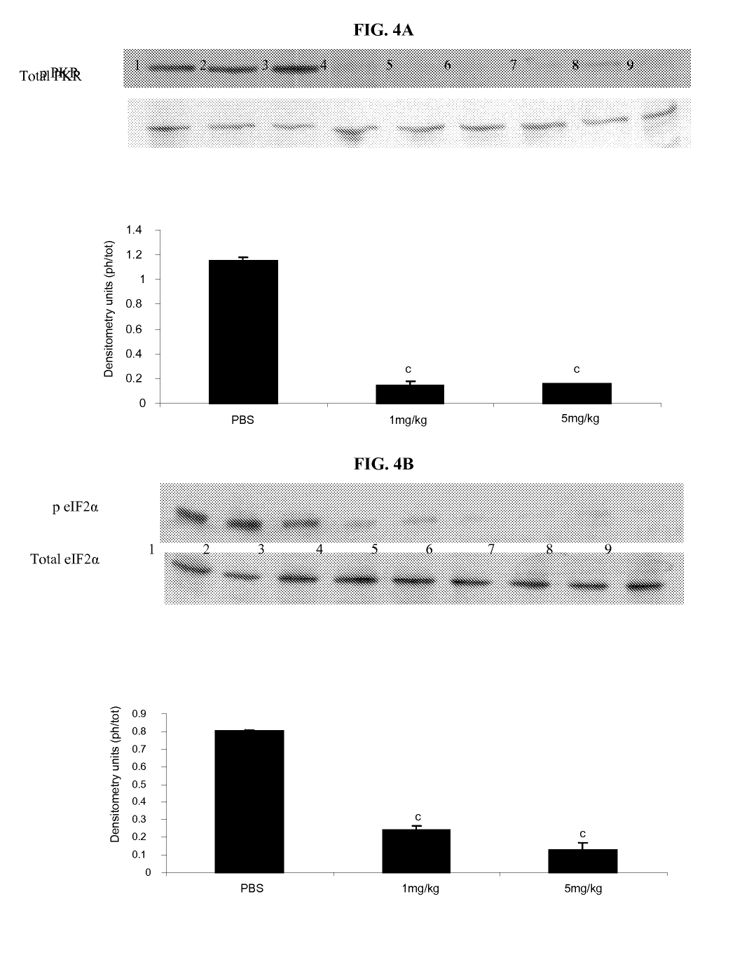

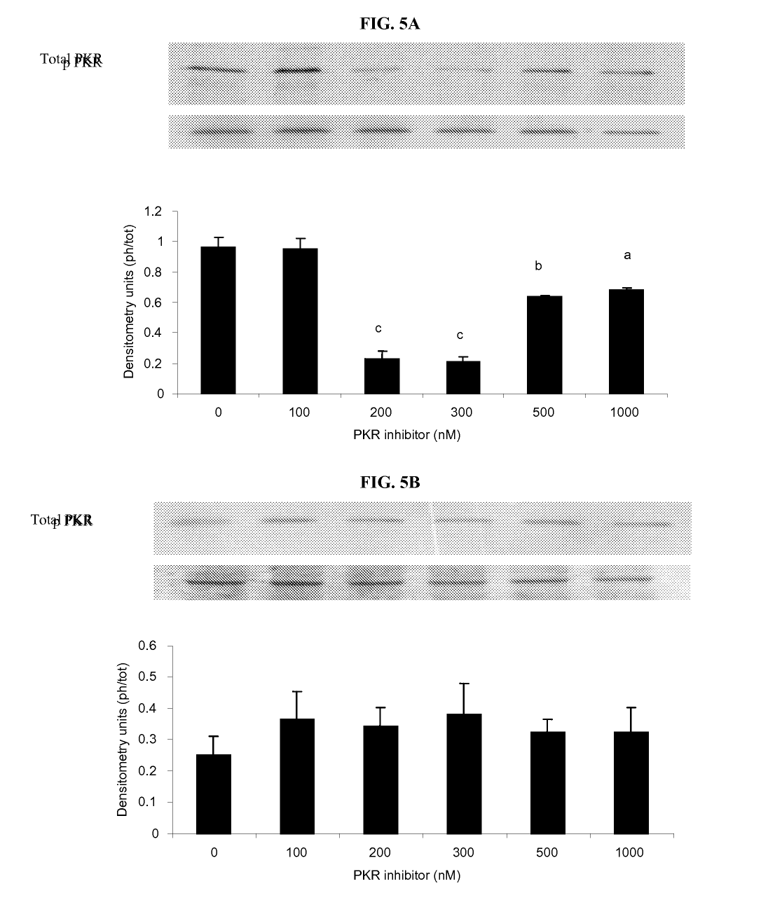

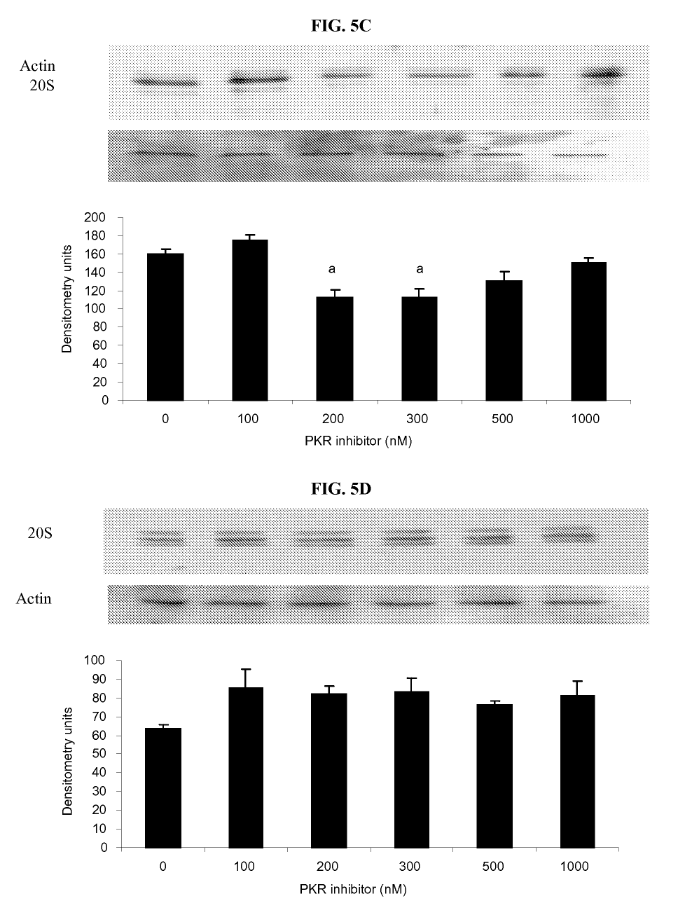

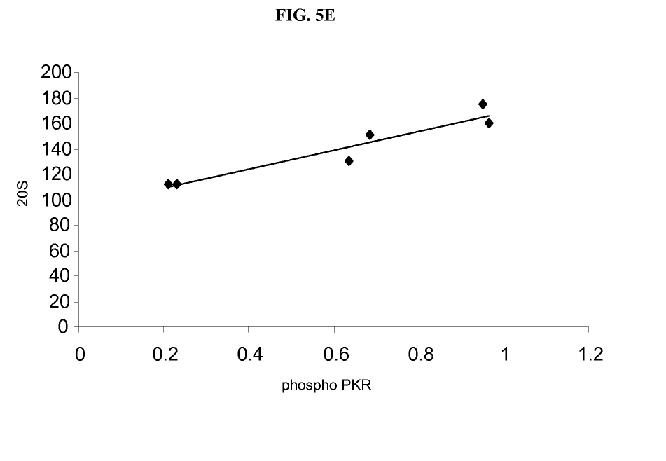

Previous studies (Eley, H. L. and Tisdale, M. J., J. Biol., 282:7087-7097, 2007) showed a low molecular weight PKR inhibitor (8-[1-(1H-imidazol-4-yl)meth-(Z)-ylidene]-6,8-dihydro-thiazol[5,4-e]indol-7-one) to attenuate the growth of the cachexia-inducing MAC16 tumor in mice. The results presented in FIG. 2 show that it also inhibited the growth of the MAC16 tumor in vitro, with a maximum effect at 200 nM, while it had no effect on the growth of the MAC13 tumor, even at a concentration up to 1000 nM. Both tumors are adenocarcinomas of the large bowel in mice, induced by prolonged administration of 1,2-dimethylhydrazine (Cowen, D. M. et al., J. Natl. Cancer Inst., 64(3):675-681, 1980), but the MAC16 induces cachexia (Bibby, M. C. et al., J Natl Cancer Inst., 78(3):539-546, 1987), while the MAC13 does not. The results in FIG. 2 show high levels of expression of both phospho PKR (FIG. 3A) and phospho eIF2α(FIG. 3B) in the MAC16 tumor, but not in the MAC13 tumor. However, the total levels of both PKR and eIF2α were similar in the two tumor types. Treatment of mice bearing the MAC16 tumor with the PKR inhibitor caused complete attenuation of the increased phosphorylation of both PKR (FIG. 4A), and eIF2α (FIG. 4B), without an effect on the total levels of PKR and eIF2α. Treatment of MAC16 cells with the PKR inhibitor produced maximum inhibition of cell growth at a concentration of 200 nM with higher concentrations producing less effective inhibition (FIG. 2). To see if this effect correlated with inhibition of autophosphorylation of PKR the effect of the inhibitor on the phospho and total PKR was determined in both MAC16 and MAC13 cells (FIG. 5). As with the solid tumors in mice MAC16 cells showed high levels of phospho PKR, while MAC13 showed very low levels. The PKR inhibitor inhibited autophosphorylation of PKR in MAC16 cells with a maximum effect between 200 and 300 nM, whilst at higher concentrations it was less effective (FIG. 5A). There was no effect of the PKR inhibitor on the low levels of autophosphorylation of PKR in MAC13 cells (FIG. 5B). In neither cell line was there an effect of the inhibitor on the total PKR in the cell. Since PKR activation has been shown to induce expression of the 20S proteasome in skeletal muscle (Eley, H. L. and Tisdale, M. J., J. Biol., 282:7087-7097, 2007), the effect of the inhibitor was determined. Both MAC16 (FIG. 5C) and MAC13 (FIG. 5D) cells expressed the 20S proteasome, but the expression was higher in MAC16 than MAC13 cells. Furthermore, the PKR inhibitor attenuated expression of the 20S proteasome in MAC16 (FIG. 5C), but not MAC13 cells (FIG. 5D). Moreover there was a linear correlation (correlation coefficient 0.957) between expression of the 20S proteasome (FIG. 5C) and expression of PKR (FIG. 5A), with the different concentrations of the PKR inhibitor (FIG. 5E), suggesting that expression of the 20S proteasome may also be controlled by expression of PKR in MAC16 cells.

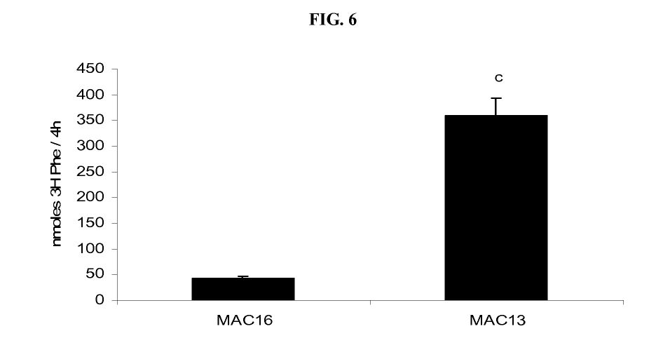

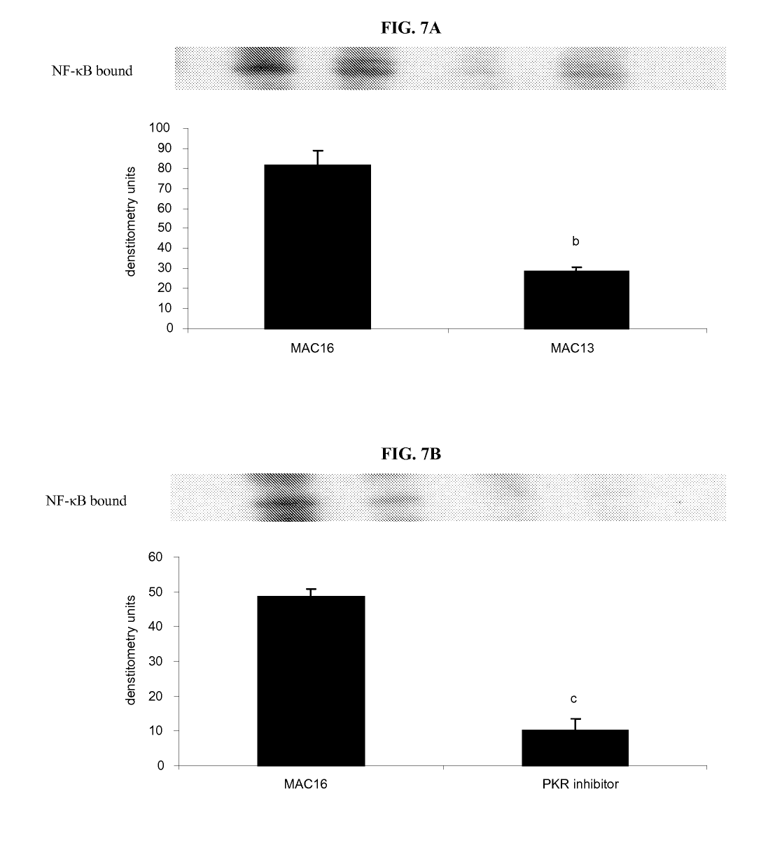

Protein synthesis in the MAC16 tumor was significantly suppressed compared with the MAC13 tumor (FIG. 6), possibly due to the increased phosphorylation of eIF2α. This suggests that phosphorylation of PKR may be important for the survival of the MAC16 tumor. One of the functions of PKR is that it is capable of activation of NF-κB (Zamanian-Daryoush, M. et al., Mol. Cell. Biol., 20:1278-1290, 2000). The data in FIG. 7A show high levels of constitutive activation of NF-κB in the MAC16 tumor, but not in the MAC13 tumor. Treatment of mice bearing the MAC16 tumor with the PKR inhibitor attenuated constitutive activation of NF-κB in the tumor, suggesting that it arose from activation of PKR.

Activation of NF-κB has been shown to play an important role in the chemoresistance of pancreatic cancer to gemcitabine (Arlt, A. et al., Oncogene, 22(21):3243-3251, 2003) and stomach cancer to 5-fluorouracil (5-FU) (Uetsuka, H. et al., Exp Cell Res., 289(1):27-35, 2003). To determine whether downregulation of NF-κB by the PKR inhibitor would increase the sensitivity of MAC16 cells to gemcitabine and 5FU the effect of the agents alone, or in combination with the PKR inhibitor (at 100 or 200 nM) on cell growth was determined (FIG. 8). 5FU alone produced significant inhibition of growth of MAC16 cells at concentrations between 1 and 10 μM, and this effect was significantly potentiated by the PKR inhibitor at both concentrations. Likewise gemcitabine induced inhibition of growth of MAC16 cells was also potentiated by the PKR inhibitors at both concentrations. These results suggest that PKR inhibitors may prove useful in the chemosensitisation of human tumors to cytotoxic agents.

### Discussion

Early studies suggested that PKR acted as a tumor suppressor, since transfection of 3T3 cells with a catalytically inactive mutant form of PKR led to cellular transformation (Koromilas, A. E. et al., Science, 257:1685-1689, 1992), while upregulation of wild-type PKR activity in M1 myeloid leukaemia cells resulted in reversal of the transformed phenotype or apoptosis (Raveh, T. et al., J. Biol. Chem., 271(41):25479-25484, 1996). However, recent studies (Kim, S. H. et al., Oncogene, 19(27):3086-3094, 2000; Yang, Y. L. et al., EMBO J., 14(24):6095-6106, 1995) cast doubt on this hypothesis. Thus PKR deficient transgenic mice are normal and do not show an increased tumor-incidence (Yang et al., 1995). In addition autophosphorylation of PKR and phosphorylation of eIF2α is between and 40-fold higher in lysates from breast carcinoma cell lines than in those from nontransformed epithelial cell lines (Kim, S. H. et al., Oncogene, 19(27):3086-3094, 2000), and is also higher in melanoma cells compared with nontransfected melanocytes in culture (Kim, S. H. et al., Oncogene, 21(57):8741-8748, 2002). In addition transformation from normal mucosa to adenomas and carcinomas of the colon was coincident with an increase in PKR expression (Kim et al, 2002). The lower PKR activity in nontransformed cell lines was partially due to lower PKR protein levels, and partially due to the presence of P58, a known cellular inhibitor of PKR (Kim, S. H. et al., 2000).

The current study shows upregulated expression of autophosphorylated PKR in tumors from mice with cachexia. Activated PKR was associated with an increased nuclear binding of NF-κB, which was attenuated by inhibition of PKR activation. Activation of NF-κB in such tumors would correlate with the clinical data showing that cachexia is a pro-inflammatory state (McMillan, D. C. et al., Nutr. Cancer, 31(2):101-105, 1998). In the murine tumor pair (MAC16/MAC13), treatment with a low molecular weight PKR inhibitor inhibited the proliferation rate of MAC16, which showed upregulation of phosphorylated PKR, but had no effect on the MAC13 tumor, which did not show activation of PKR. This result suggests that cachexia-inducing tumors, showing activated PKR, may be more susceptible to the antitumor effect of PKR inhibitors. A surprising observation was the PKR inhibitor was maximally effective in inhibiting PKR at a concentration of 200 nM, with increasing concentrations having a reduced inhibitory effect. A similar observation was made in murine myotubes in the presence of PIF (Eley and Tisdale, 2007). The PKR inhibitor is directed to the ATP-binding site in PKR, and a similar observation has been made with another ATP-binding site directed inhibitor, 2-aminopurine, in a cell-free translational assay (Jammi et al., Biochem. Biophys. Res. Commun., 308:50-57, 2003). This effect was attributed to non-specific inhibition of other components of the translational machinery. However, it is possible that higher concentrations of the inhibitor bind to PKR initiating a conformational change, which induces autophosphorylation, as it would with ATP (Lemaire, P. A. et al., J. Mol. Biol., 345(1):81-90, 2005).

Previous studies (Zamanian-Daryoush et al, 2000) have shown that PKR can activate NF-κB. PKR physically interacts, through its catalytic domain, with the upstream kinase IKK, which phosphorylates critical serine residues in IκB, leading to its degradation, releasing free NF-κB, which is then able to migrate to its specific binding sites on DNA in the nucleus. Activation of IKK by PKR appears to occur through protein-protein interactions, which stimulate the autophosphorylation of IKKβ, and not by direct phosphorylation (Bonnet, M. C. et al., Mol. Cell. Biol., 20(13):4532-4542, 2000). However, phosphorylation of eIF2 on the α-subunit has also been shown to activate NF-κB (Jiang, J. Y. et al., Mol. Cell. Biol., 23(16):5651-5663, 2003). This suggests another mechanism by which inhibition of PKR could serve to downregulate activation of NF-κB. Inhibition of constitutive activation of NF-κB by the PKR inhibitor is likely to be at least partly responsible for the inhibition of tumor growth rate. PKR mediates apoptosis induced by many different stimuli through phosphorylation of eIF2α and activation of NF-κB (Gil, J. and Esteban, M., Apoptosis. 5(2):107-114, 2000). However, PKR also activates a survival pathway, also mediated by NF-κB, which delays apoptosis (Donzé, O. et al., EMBO J., 23:564-571, 2004). Thus like NF-κB, PKR may promote tumor cell survival or death. In addition to promoting growth NF-κB enhances the angiogenic potential of tumors by increasing the expression of proangiogenic factors, such as vascular endothelial growth factor (Xiong, H. Q. et al., Int. J. Cancer, 108(2):181-188, 2004), and NF-κB-regulated gene products promote migration and invasion of cancer cells (Yebra, M. et al., Mol. Biol. Cell, 6:841-850, 1995). Although NF-κB is involved in the control of over 150 target genes, inhibition of its activation through inhibition of the autophosphorylation of PKR did not produce toxicity in mice, suggesting a new therapeutic regime for the treatment of cancer. A recent study (Kunnumakkara, A. B. et al., Cancer Res., 67(8):3853-6861, 2007) has shown that curcumin, an inhibitor of NF-κB activation, inhibits the growth of human pancreatic cancer cell lines in vitro, and potentiates the antitumor activity of gemcitabine in vivo. NF-κB has been shown to play a pivotal role in promoting gemcitabine resistance in pancreatic cancer (Arlt et al, 2003), and in the chemoresistance to 5-FU and gemcitabine in human stomach cancer cell lines (Uetsuka et al, 2003). This suggests that inhibitors of PKR may be useful in sensitizing chemoresistant tumors to chemotherapeutic agents. In the current study the PKR inhibitor has been shown to sensitize MAC16 cells to the cytotoxic effect of both 5-FU and gemcitabine, suggesting another potential therapeutic role for such agents.

Activation of PKR may explain the low rate of proliferation of some tumors, which renders them insensitive to chemotherapy and radiation. In addition to activation of NF-κB, PKR also induces phosphorylation of eIF2α, which inhibits translation initiation by competitive inhibition of the guanine nucleotide exchange factor, eIF2B, which converts eIF2.GDP into eIF2.GTP (Rowlands, A. G. et al., J. Biol. Chem., 263(12):5526-5533, 1988). However, in human breast cancer cells protein synthesis is not inhibited by the high eIF2αphosphorylation, possibly because they contain higher levels of eIF2B (Kim et al, 2000).

The results of this study show a direct relationship between the levels of phosphorylation of PKR and expression of the 20S proteasome α-subunits in the presence of the PKR inhibitor. This may provide another mechanism for tumor growth inhibition. The 26S proteasome, which is formed by combination of two 19° S. regulatory subunits with the 20S α-subunits, degrades proteins involved in cell cycle control such as p27 and p21 (Blagosklonny, M. V. et al, Biochem. Biophys. Res. Commun., 227(2):564-569, 1996). Targeted inhibition of the 26S proteasome with the dipeptide boronic acid analogue PS-341 (Velcade) has been shown to block proliferation and induce apoptosis in human pancreatic cancer cells and xenografts (Shah, S. A. et al., J. Cell Biochem., 82(1):110-122, 2001). PS-341 has also been shown to sensitize human pancreatic cancer cells to gemcitabine (Bold, R. J. et al., J. Surge. Res., 100(1):11-17, 2001). Thus inhibition of proteasome expression in tumors by inhibitors of PKR autophosphorylation may be responsible for the attenuation of tumor growth and increasing sensitivity to standard chemotherapeutic agents.

The term “about,” as used herein, should generally be understood to refer to both numbers in a range of numerals. Moreover, all numerical ranges herein should be understood to include each whole integer within the range.

It is to be understood that the invention is not to be limited to the exact configuration as illustrated and described herein. Accordingly, all expedient modifications readily attainable by one of ordinary skill in the art from the disclosure set forth herein, or by routine experimentation therefrom, are deemed to be within the spirit and scope of the invention as defined by the appended claims.

1. Composition of at least one inhibitor of double stranded RNA dependent protein kinase (PKR) and a treatment that will be enhanced by said composition.

2. A composition as claimed in claim 1 wherein said at least one inhibitor of double stranded RNA dependent protein kinase (PKR) is a phosphorylation inhibitor.

3. Composition of at least one compound to potentiate an inhibitor of double stranded RNA dependent protein kinase (PKR) and a treatment that will be enhanced by said composition.

4. A composition as claimed in claim 3 wherein said compound to potentiate an inhibitor of double stranded RNA dependent protein kinase (PKR) is a compound to potentiate a phosphorylation inhibitor of double stranded RNA dependent protein kinase (PKR).

5. A composition as claimed in claims 1 and 3, wherein said enhancement is potentiation of said treatment.

6. A composition as claimed in claims 1 and 3, wherein said enhancement is reduction of incident and/or severity of at least one side effect of said treatment.

7. A composition as claimed in claims 1 and 3, wherein said inhibitor is administered enterally or parenterally.

8. A composition as claimed in claims 1 and 3, wherein said inhibitor is a nutritional composition.

9. A composition as claimed in claims 1 and 3, wherein said inhibitor is an inhibitor of cell growth.

10. A composition as claimed in claims 1 and 3, wherein said inhibitor is an inhibitor of cell replication.

11. A composition as claimed in claims 1 and 3, further comprising at least one modifier of Protein Phosphatase 1 alpha (PPIa).

12. A composition as claimed in claim 11, wherein said PPIa dephosphorylates phosphorylated PKR.

13. A composition as claimed in claim 11, wherein said at least one modifier of PPIa is a branched chain amino acid.

14. A composition as claimed in claim 11, wherein said at least one modifier of PPIa is leucine.

15. A composition as claimed in claim 11, wherein said at least one modifier of PPIa is at least one nutritional compound.

16. A composition as claimed in claims 1 and 3 further comprising an agent to enhance delivery of a treatment to metabolically active tissue.

17. A composition as claimed in claim 16, wherein said agent is at least one of arginine and citrulline.

18. A composition as claimed in claims 1 and 3 further comprising composition of an inhibitor of cell replication.

19. A composition as claimed in claim 18, wherein nutritional composition comprises transforming growth factor-beta (TGF-β).

20. A composition as claimed in claims 1 and 3, wherein said treatment is chemotherapy.

21. A composition as claimed in claims 1 and 3, wherein said treat is radiation therapy.

22. Use of a composition as claimed in any of claim 1, 2, 3 or 4.

23. A use as claimed in claim 22, wherein said treatment is for malignancy.

24. A use as claimed in claim 22, wherein said treatment is for an autoimmune disease.

25. A use as claimed in claim 22, wherein said enhancement is potentiation of said treatment.

26. A use as claimed in claim 22, wherein said enhancement is reduction of incident and/or severity of at least one side effect of said treatment.

27. A use as claimed in claim 22, wherein said inhibitor is a nutritional composition.

28. A use as claimed in claim 22, further comprising at least one modifier of Protein Phosphatase 1 alpha (PPIa).

29. A use as claimed in claim 28, wherein said at least one modifier of PPIa is a branched chain amino acid.

30. A use as claimed in claim 1 further comprising an agent to enhance delivery of a treatment to metabolically active tissue.

31. A use as claimed in claim 30, wherein said agent is at least one of arginine and citrulline.

32. A use as claimed in claim 22 further comprising use of an inhibitor of cell replication.

33. A use as claimed in claim 32, wherein said inhibitor of cell replication, comprises transforming growth factor-beta (TGF-β).

34. A use as claimed in claim 22, wherein said treatment is chemo-therapy.

35. A use as claimed in claim 22, wherein said treat is radiation therapy.

36. Manufacture of a composition as claimed in any of claim 1, 2, 3 or 4.

37. A composition for treating a condition comprising at least one inhibitor of phosphorylation of double stranded RNA dependent protein kinase (PKR-I) and a treatment for said condition.

38. The composition of claim 37, further comprising at least one potentiatior, said at least one potentiator further enhances the inhibition of phosphorylation by said PKR-I in said mammal.

39. The composition of claim 37, wherein said condition is selected from the group consisting of cancer, cancer cachexia, anorexia, an inflammatory disease, sepsis, congestive heart failure, rheumatoid arthritis, chronic obstructive pulmonary disease, a neurodegenerative disease, an autoimmune disease, a human immunodeficiency virus infection, diabetes, a skin disease, cellular aging, Cushing Disease, rheumatic fever, and progeria.

40. The composition of claim 37, wherein said enhancement is the improvement or reduction of the severity of said condition.

41. The composition of claim 37, wherein said at least one potentiator is selected from the group consisting of an inhibitor to PKR, an analog of PKR-I, a phosphorylation inhibitor of PKR, a chemotherapeutic agent, an angiogenic agent, a vasodilatory agent, a catechin-flavanol, a bioactive protein, a branched-chain amino acid, an essential amino acid, an amino acid, an amino acid analog, a nucleotide, a vitamin, a glutamine, a sialic acid oligosaccharide, an L-theanine, a prebiotic, a probiotic, a synbiotic, an essential fatty acid, a PUFA, an MUFA, and an anti-oxidant.
